(19) United States
(12) Patent Application Publication
(10) Pub. No.: US 2026/0183543 A1
(43) Pub. Date: Jul. 2, 2026

(54) CLOSED-LOOP SPINAL CORD STIMULATION FOR MOVEMENT RESTORATION

(71) Applicant: CLOSED LOOP MEDICAL PTY LTD, Macquarie Park, New South Wales (AU)

(72) Inventors: John Louis Parker, Macquarie Park, New South Wales (AU); Gerrit Eduard Gmel, Macquarie Park, New South Wales (AU); Leonardo Silvestri, Macquarie Park, New South Wales (AU)

(21) Appl. No.: 19/131,653

(22) PCT Filed: Nov. 22, 2023
(86) PCT No.: PCT/AU2023/051190

§ 371 Date: May 21, 2025

(30) Foreign Application Priority Data
Nov. 22, 2022 (AU) 2022903532

(51) Int. Cl. A61N 1/36

(52) U.S. Cl. CPC A61N 1/36003; A61N 1/36057; A61N 1/36139

(57) ABSTRACT

Disclosed is a method for restoring movement to a spastic muscle group. The method includes measuring one or more spinal activity values in association with a movement and decoding the measured spinal activity values to determine a time-varying neural activity pattern. The method further includes performing spinal cord stimulation (SCS) of one or more afferent fibres associated with the muscle group to enable muscular activity assisting the movement, wherein the SCS is modulated according to the neural activity pattern.

The present application claims priority from Australian Provisional Patent Application No 2022903532 filed on 22 Nov. 2022, the contents of which are incorporated herein by reference in their entirety.

### TECHNICAL FIELD

The present invention relates to spinal cord stimulation for the restoration of movement to a muscle group afflicted by a movement disorder, such as for example spasticity, and in particular to dynamically modulating the applied stimulation in accordance with the movement.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

In a number of neuromodulation systems, such as those configured to provide therapeutic pain relief, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Conventional neuromodulation systems stimulate fibres in this way, for example to inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

In addition to applications for pain management, SCS also has utility in the treatment of muscle control disorders. Normal muscle tone in humans is maintained through a complex series of spinal reflexes and descending motor pathways. For example, one of the most important reflexes is through control of the spinal stretch reflex arc which is a closed neural loop that directly connects the muscle to the spinal cord via afferent (sensory) and back via efferent (motor) pathways without communication from the brain.

When a stretch reflex is activated, impulses are sent from the stretched muscle spindle via Ia afferent fibres to a corresponding alpha-motoneuron (α-MNs) of the muscle group. The α-MNs also receive input from various pathways including one descending from the brain via the dorsal column, and without this descending input or with insufficient descending input, a level of inhibition to the α-MNs may be reduced. This reduction in descending inhibition to the α-MNs in the spinal reflex arc may occur for certain muscles or muscle groups in response to an injury to the spinal cord or brain, either perinatal (e.g., cerebral palsy) or as a result of stroke. The stretch reflex arc then, in a neuroplastic response to this absence, becomes hyper-excitable for such muscle groups, keeping them in a permanent state of contraction known as spasticity. Spastic muscle groups in the limbs, in addition to being chronically painful, are of very little use for fine motor activities.

SCS has demonstrated an ability to provide relief of spasticity, and the pain associated with the condition, by stimulating nerve fibres (e.g. Aβ (A-beta) fibres) of the DC with the goal of compensating for the lack of inhibiting signals. For effective and comfortable SCS, it is necessary to maintain stimulus intensity above a threshold, such as to achieve “recruitment” of the DC nerve fibres. In almost all neuromodulation applications, response from a single class of fibre is desired, but the stimulus waveforms employed can evoke action potentials in other classes of fibres which cause unwanted side effects. It is therefore desirable to apply stimuli with intensity at a target value that does not significantly exceed the recruitment threshold, in order to avoid uncomfortable or painful percepts (e.g., due to over-recruitment of Aβ fibres).

The task of maintaining appropriate neural recruitment is made more difficult by electrode migration (change in position over time) and/or postural changes of the implant recipient (patient), either of which can significantly alter the neural recruitment arising from a given stimulus, and therefore negatively impact the ability to recruit the appropriate DC fibres to achieve relief of spasticity. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously effective stimulus regime to become either ineffectual or painful.

Many existing approaches to the application of SCS for spasticity relief are open-loop techniques in that the stimulation parameters are held fixed during the attempted recruitment of the DC fibres. A consequence is that some open-loop SCS treatment regimes, such as for cerebral palsy, are limited to an hour a day since adherence of the patient becomes impractical for longer periods (i.e., due to the level of discomfort experienced).

Moreover, the efficacy of open loop treatment depends on the stimulation intensity remaining appropriate throughout the treatment period. However, due to the aforementioned propensity for electrode migration, postural changes and/or movement of the patient, the ability to achieve DC fibre recruitment may be diminished resulting in the SCS treatment becoming ineffective or even detrimental (i.e., in the case of overstimulation events). These factors significantly impact the ability of open-loop approaches to SCS to provide effective relief of spasticity, let alone to restore some degree of voluntary movement to spastic muscle groups.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

According to a first aspect of the present technology, there is provided a method for restoring movement to a spastic muscle group, the method comprising: measuring one or more spinal activity values in association with a movement; decoding the measured spinal activity values to determine a time-varying neural activity pattern; and performing spinal cord stimulation (SCS) of one or more afferent fibres associated with the muscle group to enable muscular activity assisting the movement, wherein the SCS is modulated according to the neural activity pattern.

In some embodiments, measuring the spinal activity values comprises measuring non-evoked potentials at the spinal cord.

In some embodiments, the non-evoked potentials are sensory afferent responses associated with the movement as it occurs.

In some embodiments, the non-evoked potentials are efferent potentials associated with the movement.

In some embodiments, measuring the spinal activity values comprises measuring activity using an accelerometer.

In some embodiments, the neural activity pattern is a time series of values representing a degree of relative excitability of a stretch reflex of the muscle group at discrete sample times of the movement.

In some embodiments, the neural activity pattern is a sequence of excitation response (ER) values.

In some embodiments, the ER values are determined from an induced H-reflex response of the muscle group.

In some embodiments, the ER values are determined from a growth curve of the H-reflex response of the muscle group.

In some embodiments, decoding the measured spinal activity values comprises: (i) extracting, from the spinal activity values, one or more motor pattern feature values associated with the movement; and (ii) determining the neural activity pattern by applying the one or more motor pattern feature values to one or more spinal activity models.

In some embodiments, motor pattern feature values include corresponding spinal activity values.

In some embodiments, motor pattern features include one or more of: a cycle period, a burst duration, a duty cycle, and a phase of an excitation of an individual muscle of the muscle group.

In some embodiments, at least one of the spinal activity models is a movement-specific model trained with a set of training spinal activity values obtained from individuals performing the movement.

In some embodiments, training the movement-specific model comprises: identifying one or more gait phases of the movement; extracting training motor pattern feature values from the training spinal activity values; and generating, from the training motor pattern feature values, model parameters representing expected motor pattern feature values for the one or more gait phases of the movement.

In some embodiments, the movement-specific model is a two-tier neural network classifier with parameters generated by processing an input sequence of the training motor pattern feature values.

In some embodiments, the movement-specific model is a hidden Markov model with parameters generated by processing an input sequence of the training motor pattern feature values.

In some embodiments, training the movement-specific model comprises: processing the training spinal activity values to generate a spinal activity graph; and determining representative inhibitory and excitatory response levels from the spinal activity graph.

In some embodiments, the representative inhibitory and excitatory response levels are threshold values determined by identifying local maxima and local minima in the spinal activity graph.

In some embodiments, determining the neural activity pattern comprises: determining, based on the representative inhibitory and excitatory response levels, one or more periods of relative excitation or inhibition associated with performing the movement.

In some embodiments, determining the neural activity pattern further comprises processing the one or more periods of relative excitation or inhibition to determine one or more gait phases of the movement.

In some embodiments, performing the spinal cord stimulation includes: (i) determining a recruitment target level (RTL) by processing the neural activity pattern; (ii) applying a stimulus to the spinal cord to stimulate the one or more afferent fibres associated with the muscle group; (iii) measuring an intensity of a neural response evoked by the stimulus; and (iv) adjusting an intensity of a subsequent stimulus applied by step (ii) based on a feedback signal representing a difference between the measured neural response intensity and the determined RTL, wherein steps (i) to (iv) are repeated such that the determined RTL varies over time in accordance with corresponding values of the neural activity pattern.

In some embodiments, the steps (ii) to (iv) are repeated over a number of cycles for each determined RTL of step (i), wherein the number of cycles is a predetermined threshold number.

In some embodiments, the steps (ii) to (iv) are repeated in one or more cycles until the feedback signal is within an error tolerance value for each determined RTL of step (i).

In some embodiments, the response model is a linear regression function that is specific to the one or more afferent fibres.

In some embodiments, in response to the neural activity pattern comprising one or more excitation response values determined from an induced H-reflex response of the muscle group, determining the RTL involves using the determined excitation response values as input to a response model.

In some embodiments, the one or more afferent fibres are determined from the neural activity pattern.

According to a second aspect of the present technology, there is provided a system for restoring movement to a spastic muscle group, the system comprising: a stimulator including an electrode array and a pulse generator, the stimulator configured to: measure one or more spinal activity values in association with a movement; and apply, via the electrode array, stimuli to the spinal cord to stimulate one or more afferent fibres associated with the muscle group; and a processor configured to: decode the one or more spinal activity values to determine a time-varying neural activity pattern; and control the stimulator to apply the stimuli to the one or more afferent fibres to enable muscular activity assisting the movement, wherein the stimuli are modulated according to the neural activity pattern.

In some embodiments, the electrode array comprises one or more leads implanted proximate to the afferent fibres.

In some embodiments, the one or more afferent fibres comprise Aβ afferent fibres.

In some embodiments, one of the one or more leads is implanted above the midline of the dorsal column.

In some embodiments, the one or more afferent fibres comprise Ia afferent fibres.

In some embodiments, one of the one or more leads is implanted over the dorsal roots of the dorsal column.

In some embodiments, the processor is part of the stimulator.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 is a schematic illustrating an implanted spinal cord stimulator, according to one implementation of the present technology;

FIG. 2 is a block diagram of the stimulator of FIG. 1;

FIG. 3 is a flow diagram of a method for restoring movement to a spasticity-afflicted (spastic) muscle group of a patient according to one aspect of the present technology;

FIG. 4a is a schematic of an exemplary configuration of electrode arrays of the stimulator of FIG. 1 for restoring movement to a spastic muscle group according to one aspect of the present technology;

FIG. 4b is a graph of the typical form of an evoked compound action potential (ECAP);

FIG. 4c is an illustration of an idealised activation plot for one posture of a patient undergoing neural stimulation;

FIG. 5 is a flow diagram of a method for decoding spinal activity values to generate the time-varying neural activity pattern according to one aspect of the present technology;

FIG. 6a is an illustration of a cross-section of the spinal cord showing the spinal reflex arc;

FIG. 6b is an illustration of the stretch reflex of a lower limb muscle group;

FIG. 6c is a schematic diagram illustrating an example of a locomotor control system according to one aspect of the present technology;

FIG. 7a is a flow diagram of a method for training a spinal activity model corresponding to a given movement, according to one aspect of the present technology;

FIG. 7b is an illustration of an example of training a spinal activity model in the form of a Hidden Markov Model (HMM) trained by the method of FIG. 7a;

FIG. 7c is an illustration of an example of training a spinal activity model in the form of a two-tier neural network classification model trained by the method of FIG. 7a;

FIG. 8a is a flow diagram of a method for training a spinal activity model including a spinal activity graph, according to one aspect of the present technology;

FIG. 8b is an illustration of an exemplary spinal activity graph trained by the method of FIG. 8a;

FIG. 9 is a flow diagram of a method for performing modulated SCS via a neuromodulation device for restoration of movement, according to one aspect of the present technology; and

FIG. 10 is a block diagram of a neuromodulation system configured to perform the method of FIG. 9.

### DETAILED DESCRIPTION OF THE PRESENT TECHNOLOGY

As an alternative to the open-loop approaches described above, performing SCS with closed-loop control enables the adjustment of the stimulation parameters to maintain a predetermined level of neural recruitment. Implementing closed-loop control has demonstrated the ability to address some of the drawbacks of open-loop SCS in the context of therapeutic pain management. Closed-loop control of an applied stimulus (i.e., a stimulus signal) is dependent on the ability to accurately measure the intensity of a neural response evoked by the stimulus (i.e., as a neural response signal). The neural response signal is measurable in terms of the action potentials generated by the depolarisation of a large number of fibres by the stimulus to form an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches for obtaining a neural response measurement are described by the present applicant in International Patent Publication No. WO2012/155183, the content of which is incorporated herein by reference. In the context of relieving spasticity, the closed-loop control of a stimulus has been clinically shown to enable a much higher level of DC activation (nearly 10 times the average activation), and as a result has the potential to provide significantly greater descending inhibition thereby improving the potential to treat muscle spasticity (see Parker and Dietz [1]). Significantly, in theory, a closed-loop SCS system will enable real-time instantaneous control of the level of DC fibre recruitment, even during postural change.

There have been recent advances in the use of closed-loop SCS for treating spasticity. For example, in International Patent Application No. PCT/AU2023/051112, the contents of which are incorporated herein by reference, systems and methods are proposed for determining a target neural response value, referred to herein as the “recruitment target level” (RTL), enabling closed-loop control of a corresponding stimulus applied to the spinal cord to treat spasticity in a muscle, or muscle group. This enables a closed-loop SCS system to be programmed to relieve spasticity in the muscle by the closed-loop control of the stimulus intensity to maintain fibre recruitment at the desired level.

Despite the promise of closed-loop SCS, these approaches are still limited in that the therapeutic benefit is focused on alleviating the hyper-excited state of the spastic muscle, or muscle group. That is, “relief” of spasticity, in the sense of addressing persistent hyper-excitability of the muscle response (i.e., by compensating for a lack of descending inhibition), still leaves the motor control function of the patient compromised. Accordingly, even if treatments to relieve spasticity are successful, there is still a desire to restore full or partial movement of the muscle group.

The brain contains most of the neural circuitry that controls voluntary movement. Accordingly, existing approaches for the restoration of movement in the presence of a motor control disorder are based on an analysis and/or compensation of the relevant brain signals. Specifically, voluntary movement is initiated and planned in supraspinal centres in the brainstem and midbrain. Although these areas may be damaged in instances of motor control disorder (e.g., in individuals with cerebral palsy), the rest of the neural circuitry involved in movement remains intact. This includes the localized neuronal central pattern generators (CPGs), which activate and coordinate the spinal alpha motor neurons (α-MNs), which in turn directly control the contraction of skeletal muscles involved in voluntary stereotyped, rhythmic movement such as locomotion.

In healthy individuals, the output of the efferents (α-MNs or motor fibres) connected to muscles is regulated in parallel by the CPGs and multiple afferents (sensory fibres) from the skin and muscles. As a result, the corresponding mechanisms of the spinal reflex arc provide a theoretical basis for restoration of movement by controlling the degree of inhibition (and therefore excitability) of the stretch reflex of the given spastic muscle group.

SCS provides an ability to recruit nerve fibres to achieve a controlled degree of inhibition of the stretch reflex, as performed to relieve spastic muscles. However, there are several difficulties faced in the design of SCS systems and methods that are able to address the problems associated with achieving full movement restoration. First, previously proposed approaches to closed-loop SCS perform static recruitment of Aβ neurons by targeting a neural response that is a fixed value (e.g., a predetermined ECAP amplitude) in order to reduce the excitability of the stretch reflex by a predetermined amount. As discussed, while potentially effective to relieve spasticity, this alone is inadequate to restore movement.

Second, restoring full or partial motor control requires selectively inducing an inhibitory or excitatory effect in the stretch reflex of the muscle group, where the effect varies over time based on the intended or actual movement of the individual. This is significant because voluntary movement signalling originates in the brainstem and motor cortex, in contrast to SCS which relies on the application of stimuli and the detection of corresponding neural responses at the spinal cord. It is desired to ameliorate these drawbacks, or one or more other deficiencies of the previous approaches, or to at least provide a useful alternative.

### Overview

Disclosed herein are methods and systems for performing closed-loop SCS to restore movement to a muscle group afflicted by a movement disorder, such as spasticity, generally involving: (i) identifying an intended or actual movement based on the measurement and interpretation of movement-related signals from the spinal cord or elsewhere; and (ii) modulating the operation of the closed-loop SCS to enable the spastic muscle group to perform the intended or actual movement, or at least assist the same. Various embodiments are described in accordance with the aforementioned general approach in which a signal (a “spinal activity signal”) is measured from the spinal cord as one or more electrical field parameter values (one or more “spinal activity values”). The spinal activity values are associated with an intended or actual movement involving the muscle group. The spinal activity values are decoded or interpreted to determine a time-varying neural activity pattern. The neural activity pattern is related to the expected level of inhibition or excitation of the stretch reflex by the muscle group to perform the movement. SCS is applied to cutaneous afferents, such as Aβ fibres or Ia fibres, associated with the muscle group to enable muscular activity assisting the movement, where the intensity of stimulus of the SCS is modulated according to the neural activity pattern.

The spinal activity values may be measured as non-evoked potentials, such as non-evoked CAPs or EMGs. The non-evoked potentials may be descending efferent potentials associated with the intended movement, or sensory afferent responses associated with the actual movement. Such sensory afferent responses may be activations of cutaneous and/or proprioceptive sensory fibres as a result of the movement. Features are obtained from the spinal activity values, to represent rhythmic electrical activity associated with locomotion in the spinal cord (referred to as “motor pattern features”). That is, the motor pattern features are a spinal representation of the α-MN potentials for performing muscular activity assisting the movement. Various metrics may be used as components of the motor pattern features. In the described embodiments, the spinal activity values are used directly (or in a pre-processed form) as corresponding motor pattern feature values. In other embodiments, the motor pattern features may include one or more other features that characterize rhythmic muscle activity such as a cycle period, burst duration, duty cycle, and phase of firing of the responses for an individual muscle.

In other implementations, spinal activity values may be provided by an accelerometer, or another motion-tracking device, mounted on, or implanted within, the spastic muscle group to measure the activity of the muscle group directly during the movement.

The neural activity pattern represents a degree of relative excitability of the stretch reflex of the muscle group performing the muscular activity over time. For example, the neural activity pattern may be represented as a time series of values of relative excitability at discrete sample times of the movement. In the described embodiments, the neural activity pattern is represented as a sequence of excitation response (ER) values. In one approach to determining the neural activity pattern, the ER values are derived from an H-reflex of the muscle group across a range of intensities of separately applied probe stimuli. The H-reflex is an artificial emulation of the stretch reflex that is triggered not by a stretching of the muscle spindle but by stimulation of the Ia afferent fibres over which the signal from the muscle spindle would travel. The H-reflex has been used to characterise the excitability of the stretch reflex.

In some embodiments, decoding measured spinal activity values involves extracting, from the measured spinal activity values, one or more motor pattern feature values associated with the movement. The neural activity pattern is determined by applying the motor pattern feature values to at least one spinal activity model. The spinal activity model captures the expected motor pattern features of an individual (e.g., the patient or a healthy individual) performing the movement via a model training process. In some embodiments, the spinal activity model is movement-specific, and represents expected spinal activity over the movement duration. The movement is characterized as occurring over one or more movement phases (i.e., stages of a gait cycle) in which the stretch reflex experiences a particular degree of relative excitability.

In some embodiments, the spinal activity of the intended or actual movement is modelled as a stochastic process over the time-varying movement phases, for example as a movement-specific Hidden Markov Model (HMM). This enables the neuromodulation system to autonomously select an appropriate activity model corresponding to the intended or actual movement from a plurality of candidate movements (each with a corresponding trained HMM). Therapy may therefore be performed for restoration of a variety of movements dynamically without requiring an indication of the movement.

In other embodiments, the spinal activity of the intended or actual movement is modelled by the application of machine learning techniques to the training motor pattern feature (or spinal activity) data to produce an indication of the movement phases. For example, a two-tier neural network classifier, including dynamic recurrent neural network (DRNN) and artificial neural network (ANN) sub-units, may be trained to provide output indications of gait phase (e.g., for a walking movement) based on input motor pattern feature (or spinal activity) data. The determined movement phases are mapped to a corresponding neural activity pattern (i.e., a time-varying sequence of ER values) thereby enabling modulated closed-loop SCS.

In another approach, the neural activity pattern is derived from a spinal activity graph determined from amplitudes of the non-evoked potentials of the spinal activity values. Representative inhibitory and excitatory response levels of the muscular activity are determined from the spinal activity graph. Mapping the time-varying non-evoked potential amplitude values of the graph to the representative inhibitory and excitatory response levels (i.e., across the movement phases) enables a determination of the neural activity pattern as a quantification of a degree of relative excitation or inhibition of the stretch reflex.

Advantageously, since the representative inhibitory and excitatory response levels can be determined directly from the values of each graph (e.g., using maxima and minima detection), the computational requirements of this “graph-based spinal EEG” approach are reduced compared to non-analytical approaches (e.g., machine learning or pattern classification). Furthermore, model training may be performed on the patient (i.e. with the spastic muscle group for which movement is being restored) eliminating the need to conduct separate training or evaluation activities with a set of healthy individuals.

A form of closed-loop SCS is performed to restore movement to the muscle group by modulating the intensity of an applied stimulus based on the neural activity pattern. The applied stimulus is controlled via a feedback signal determined by comparing a measured neural response intensity value evoked by the stimulus (e.g., an ECAP) to a corresponding target value. The target value represents a desired degree of recruitment of the Aβ or Ia afferent fibres, referred to as the “recruitment target level” (RTL).

The RTL may be determined from the ER values of the neural activity pattern, and therefore varies dynamically over time in synchrony with the intended or actual movement. For example, an ER-RTL mapping may be derived from a set of neural responses and corresponding ECAP values obtained from healthy individuals (i.e., with no movement disorder afflicting the muscle group) as part of a validation trial conducted prior to the movement restoration therapy. Alternatively, the ER-RTL mapping may be derived from a set of neural responses and corresponding ECAP values obtained from the patient, or one or more other individuals with the same or similar disordered movement. In some embodiments, individual ER values of the neural activity pattern are input to a pre-determined response model to produce the RTL as output in real-time, or substantially real-time. In other embodiments, the RTL may be derived directly from the non-evoked potential values of the spinal activity (e.g., by determining amplitudes of the sensory afferent responses associated with the actual movement).

Decoding measured spinal activity values to corresponding mapped neural activity patterns using time-dependent models of movement provides an approach to restoring movement without requiring the capture or analysis of brain signals. Measured activity of the spinal circuits (i.e., the spinal activity values) is decoded to determine an intention of a movement, or an actual movement, by comparing it with a model of the expected activity resulting from performance of the movement (e.g., in healthy individuals). The movement is characterized in terms of a neural activity pattern of expected excitability of the stretch reflex for proper control of a muscle group involved in the movement. These spinal CPG modelling aspects are utilized to advantageously restore movement to spastic muscles via the additional aspect of (iii) modulating SCS applied to the cutaneous afferents in accordance with the time-varying neural activity pattern for the movement (e.g., represented as a time-varying series of excitation response values).

Although the embodiments described herein provide restoration of movement in the context of muscle groups afflicted by spasticity, it will be appreciated that the relevant systems, methods, and devices are equally applicable to alleviate the effects of other movement disorders for which the underlying mechanisms of the spinal reflex arc provide a means for achieving control of the muscle groups.

### System for Restoration of Movement to Spastic Muscles

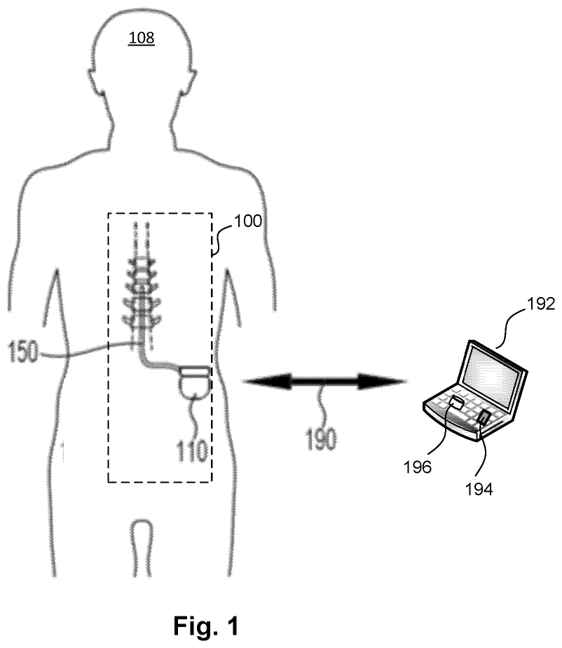

FIG. 1 schematically illustrates an embodiment a spinal cord stimulator 100, depicted as implanted in a patient 108, and a user device 192 that is external to the stimulator 100. The stimulator 100 and the user device 192 are collectively configured as part of a neuromodulation system for restoring movement to a spastic muscle group via modulated closed-loop spinal cord stimulation (CL-SCS).

Stimulator 100 comprises an electronics module 110 implanted at a suitable location. Stimulator 100 further comprises an electrode array 150, depicted as implanted within the epidural space, and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Stimulator 100 operates as a neural modulation device that performs CL-SCS by: applying a stimulus to the spinal cord to stimulate one or more nerve fibres; and measuring a neural response signal as a set of neural responses evoked in response to the stimulus. For example, a neural response may be measured as a compound action potential (CAP) that is evoked in response to the stimulus (referred to as an “ECAP”). An ECAP typically has a maximum amplitude in the range of microvolts, whereas an applied stimulus signal evoking the CAP is typically several volts. Stimulator 100 is also configured to measure, via the electrode array 150, a spinal activity signal as one or more values of non-evoked compound action potentials generated in the epidural space in association with an intended or actual movement of patient 108.

Stimulator 100 is operable in a closed loop mode in which the intensity of the applied stimulus (e.g., the amplitude of a corresponding stimulus signal) is adjusted in response to a feedback signal. The feedback signal is determined from a difference between values of the measured neural response signal and a target value of the closed loop, such as the RTL in the embodiments discussed herein. This operation may also be referred to as closed loop neural stimulation (CLNS).

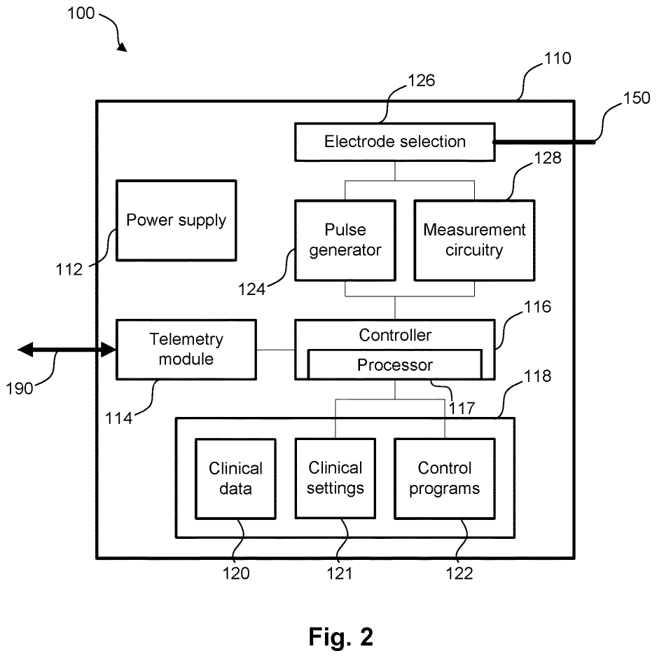

FIG. 2 is a block diagram of the stimulator 100. Electronics module 110 contains electronic components enabling the operation of stimulator 100. Electronics module 110 includes a battery, or other power supply, 112 and a telemetry module 114. In implementations of the present technology, any suitable type of communications channel 190, such as infrared (IR), radiofrequency (RF), capacitive and inductive transfer, may be used by telemetry module 114 to transfer power and/or data to and from the electronics module 110 via communications channel 190.

Module controller 116 has an associated memory 118 storing one or more of clinical and/or program data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Controller 116 includes a processor 117 configured to execute one or more machine readable instructions of the control programs 122. The control programs 122 may include software programs written in a programming language such as C++ or Java, and configured, on execution, to instruct the processor 117 to perform the operations of method 300, or the associated sub-processes and methods.

Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

The user device 192 is a computing device operable by a user, such as a clinician or the patient 108. In some embodiments, the user device 192 is a mobile computing device, such as a smart phone or tablet. In alternative embodiments, the user device 192 may be implemented as one or more full-scale computer devices, such as an Intel Architecture computer system configured as a desktop or laptop workstation.

In an exemplary configuration, user device 192 includes a processor 194 in communication with a memory system 196. The user device 192 further includes a networking system, one or more display interfaces, and one or more I/O device interfaces (not shown). The processor 194 may be any microprocessor which performs the execution of sequences of machine instructions, and may have architectures consisting of a single or multiple processing cores such as, for example, a system having a 32- or 64-bit Advanced RISC Machine (ARM) architecture (e.g., ARMvx). The processor 194 issues control signals to other device components via a system bus, and has direct access to at least some forms of the memory system 196.

Memory system 196 includes internal storage media for the electrical storage of machine instructions required to execute one or more software or firmware modules. For example, the internal storage media may include a combination of random access memory (RAM), non-volatile memory (such as ROM or EPROM), cache memory and registers, and high volume storage subsystems such as hard disk drives (HDDs), or solid state drives (SSDs). The modules stored in the memory system 196 include, but are not limited to, an operating system and one or more local application programs. For example, the local application programs may include, in some embodiments, programs for performing the operations of method 300, or the associated sub-processes and methods.

The user device 192 is connectable to one or more other computing devices and/or electronic modules via the networking system. A communications channel 190 connects the user device 192 to the module 110 of the stimulator 100. The communications channel 190 includes a wireless or wired transmission media enabling the exchange of data between the user device 192 and the module 110. The communications channel 190 may be implemented as a transcutaneous channel. Communications channel 190 may be active on a substantially continuous basis, at periodic intervals, at non-periodic intervals, or upon request from the device 192.

The stimulator 100 is programmable by the user device 192. In some embodiments, the user device 192 transmits data to the stimulator 100 to configure one or more of the control programs 122, that when executed by processor 117 control the operation of the stimulator 100. User device 192 may thus provide a clinical interface to configure the operation of the implanted stimulator 100 and recover data stored on the implanted stimulator 100, either as generated from the execution of the control programs 122 or otherwise. This configuration is achieved by program instructions collectively referred to as the Clinical Programming Application (CPA) and stored in an instruction memory of the clinical interface.

In one implementation, the processor 117 controls the operation of the implanted stimulator 100 according to one or more programs including: a spinal activity detection program; a decode spinal activity program; and a modulated CL-SCS program.

The modulated CL-SCS program controls the stimulator 100 to perform CL-SCS to enable muscular activity in a muscle group of the patient 108, thereby assisting a particular movement of the patient 108. Under the operation of the modulated CL-SCS program, the stimulator 100 is configured to repeatedly: determine, via the controller 116, a recruitment target level (RTL) by processing the values of a time-varying neural activity pattern of the movement; apply a stimulus at a specified intensity via the operation of the pulse generator 124 and electrode array 150; measure the corresponding neural response signal comprising one or more neural responses via the electrode array 150; and adjust, via the controller 116, an intensity of a subsequent stimulus based on a feedback signal determined from a difference between the one or more neural responses and the RTL, where the determined RTL varies over time in accordance with corresponding values of the neural activity pattern.

The processor 117 is configured to control the operation of the implanted stimulator 100 by selectively executing the spinal activity detection program. Processor 117 operates the electrode array 150 to measure values of non-evoked CAPs over a duration of time (i.e., an activity detection period). Processor 117 receives the one or more measured spinal activity values and, in some embodiments, performs data processing and/or storage operations on the values (e.g., to store the values in memory 118).

In a therapy mode of operation, the processor 117 is configured to control the operation of the implanted stimulator 100 by selectively executing the spinal activity decoding program. The spinal activity decoding program determines, from a spinal activity signal of patient 108, a time-varying neural activity pattern representing a degree of relative excitability of the stretch reflex of a muscle group for carrying out a movement by the patient 108. The spinal activity decoding program generates, as an output, a set of excitation response values and a set of time intervals enabling the execution of the modulated CL-SCS program to restore the movement for the patient 108.

The user device 192 is configured to provide the stimulator 100 with parameters for the execution of the spinal activity detection, spinal activity decoding, and modulated CL-SCS programs in the therapy mode. The parameters may include, for example: therapy parameters, defining one or more values or settings to apply or adjust the stimulus and to measure the corresponding neural response; and model parameters for one or more spinal activity models, defining expected motor pattern values, expected ER values (and/or corresponding RTLs), and time interval values of movement phases in which the expected ER values and/or RTLs are to be utilized in the SCS delivered by the modulated CL-SCS program. In some embodiments, the spinal activity model parameters are obtained by training and/or experimentation activities conducted prior to movement restoration therapy administered to the patient 108 during therapy mode operation of the stimulator 100.

In an evaluation mode of operation of the stimulator 100, the processor 117 transmits the measured spinal activity values to the user device 192 via channel 190. The user device 192 is configured to process the measured spinal activity values, for example to perform spinal activity model training and/or to store the spinal activity values.

In some embodiments, one or more steps of the spinal activity detection, spinal activity decoding, and/or the modulated CL-SCS programs are performed in response to control signals and/or data received from the user device 192. For example, the stimulator 100 may be configured to receive a control signal from the user device 192, via the communications channel 190, providing an instruction to commence or cease execution of one or more of the programs, and/or any other of control programs 122.

In some embodiments, the execution of the control programs 122 by the processor 117, such as for example the modulated closed-loop SCS (CL-SCS) program and the spinal activity detection program, occurs in response to control instructions received from the user device 192, or another device (e.g., a remote controller of the stimulator 100).

In other embodiments, the processor 117 is configured to execute the control programs 122 automatically and/or autonomously. This enables the configuration and execution of methods for SCS-based therapy to restore movement to muscle groups afflicted with spasticity “on-line”, and in real-time, by the stimulator 100. Further, the stimulator 100 may operate self-sufficiently to repeatedly identify intended or actual movements of the patient 108 (i.e., by detecting and decoding spinal activity values), and to perform modulated CL-SCS to restore the movement (i.e., without further instruction or communication from the user device 192 once initially programmed).

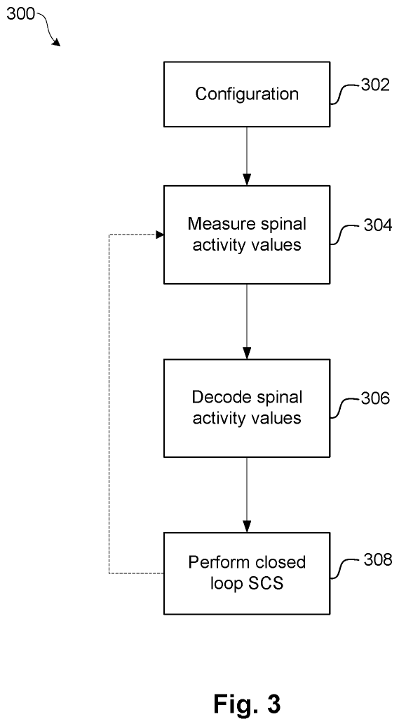

FIG. 3 illustrates a method 300 performed by a neuromodulation system for restoring movement to a spastic muscle group of a patient according to one aspect of the present technology. The application of modulated closed-loop SCS (i.e., at step 308) is enabled by measuring spinal activity in the patient (i.e., at step 304) and subsequently decoding the measured spinal activity to determine and characterize an intended or actual movement (i.e., at step 306). By programming the modulated CL-SCS with a time-varying recruitment target level, which is determined according to a characteristic neural activity pattern of the determined movement, inhibition or excitation of the stretch reflex of the spastic muscle group can be modulated to restore the movement.

### Configuration

At step 302, the stimulator 100 is configured for operation to restore movement to a muscle group of patient 108. The spinal cord stimulator 100 is implanted in patient 108, according to one implementation of the present technology. In one implementation, stimulator 100 is implanted in the patient's lower abdominal area or posterior superior gluteal region. In other implementations, the electronics module 110 is implanted in other locations, such as in a flank or sub-clavicularly.

Electrode array 150 includes one or more electrodes that are collectively positioned to enable stimulation of at least one afferent fibre in the dorsal column associated with the spastic muscle group, and measurement of one or more corresponding neural responses evoked by the stimulation, as described below. During operation, the stimulator 100 is configured to cause one or more electrodes (“stimulus electrodes”) of the electrode array 150 to apply an electrical pulse to the dorsal column (DC) via activation of the pulse generator 124. The activation of the pulse generator 124 is controlled by controller 116, which is configurable to cause the generation of the applied pulse at a specified intensity. For example, the applied pulse may be a current pulse with the intensity corresponding to the pulse amplitude. The applied pulse causes the depolarisation of neurons, and generation of propagating action potentials thereby stimulating the nerve fibres. Delivery of an appropriate stimulus (i.e., of sufficiently high intensity) to the nerve evokes a neural response comprising an evoked compound action potential (ECAP). The stimulus electrodes are configurable to deliver stimuli periodically at any suitable frequency, for example 30 Hz, although other frequencies may be used including frequencies as high as the kHz range.

The neural response is detected by the measurement of an electrical field parameter by the measurement circuitry 128 components. For example, the electrical field parameter may include at least one of: an evoked neural compound action potential (ECAP); a non-evoked neural compound action potential (nECAP); a local field potential (LFP); a slow response; or another physiological parameter (such as EMG, ECoG, and EKG). In the described embodiments, the stimulator 100 is configured to measure the intensity of neural responses in the form of ECAPs propagating along the target nerve fibres.

Stimulus electrodes are positioned in the dorsal epidural space above the DC to achieve preferential recruitment of afferent fibres associated with the muscle. In the described embodiments, the therapeutic stimulation and measurement are localised to the DC, as performed by one or more electrodes of array 150 that are positioned in the dorsal epidural space. Electrode selection module 126 determines a configuration of electrodes that are configured for delivering the applied stimulus to nerve fibres of the muscle group (“stimulus electrodes”) and electrodes that are configured to measure the neural response evoked by the applied stimulus (“measurement electrodes”).

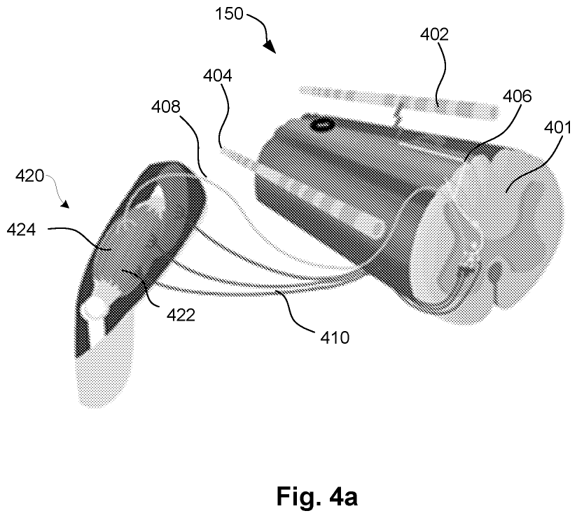

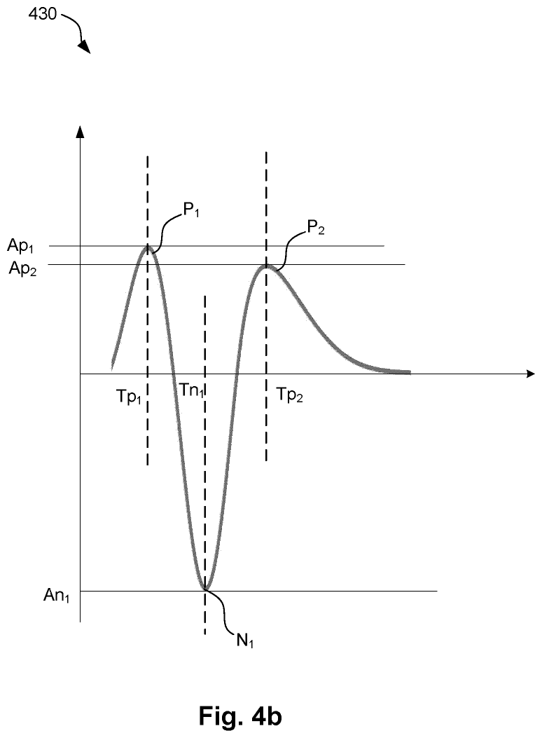

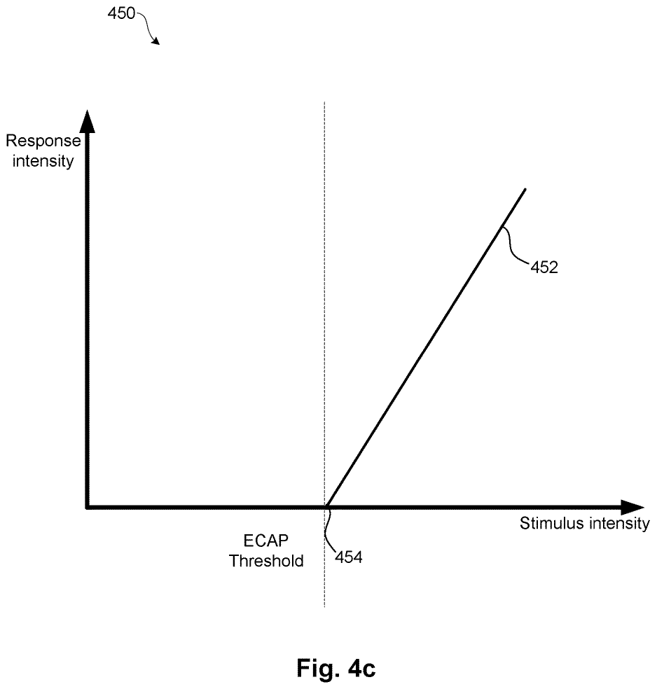

FIG. 4a illustrates an exemplary configuration of electrode array 150 implanted at the DC for therapeutic SCS to restore movement to a spastic muscle group 420. For the embodiments described herein, muscle group 420 is a muscle group of the lower limb (leg) of patient 108, including a flexor muscle 422, and an extensor muscle 424.

The array 150 incudes a first lead 402 positioned above the midline of the DC 401 and a second lead 404 positioned laterally to the first lead 402 over the dorsal roots of the DC 401. The first lead 402 is configured to apply stimulation to the Aβ fibres 406 associated with the muscle group 420 to inhibit the stretch reflex (as described below), and to measure the evoked neural responses in the DC 401. The neural activity may be related to a movement associated with the muscle group 420.

The second lead 404 is positioned to positioned to apply stimulation to, and enable the recruitment of, the Ia afferent fibres 408 associated with the muscle group 420 to excite (as opposed to inhibit) the stretch reflex. Also shown in FIG. 4a are the efferent fibres 410 associated with the direct muscle response of at least the flexor muscle 422 of the muscle group 420. In the configuration illustrated by FIG. 4a, both the first (402) and the second (404) leads are configured to both deliver the applied stimulus and measure the corresponding neural response (i.e., to act as both stimulus and measurement leads).

Electrode array 150 further includes one or more spinal activity electrodes (not shown) configured to measure electrical field parameter values representing non-evoked spinal activity. That is, the spinal activity electrodes measure the rhythmic electrical activity (represented as a CAP amplitude values) produced by the CPG and associated with an intention of patient 108 to perform a movement, or with the actual movement itself.

In some embodiments, the spinal activity electrodes are implanted, or otherwise located subcutaneously, at the lumbar spinal region. In other embodiments, the spinal activity electrodes measure non-evoked CAP values via the detection of a peripheral response such as an EMG (i.e., by placing the spinal activity electrodes on the skin). In yet other embodiments, an accelerometer may be mounted on the skin of the muscle group to provide the spinal activity data. This enables a stimulator 100 to be configured to measure spinal activity of an individual without implantation of the electrode array 150 or module 110. This is advantageous to enable the neuromodulation system to collect spinal activity values from healthy individuals during validation tests (e.g., to generate training data for spinal activity models, as described below).

FIG. 4b illustrates the typical form of an ECAP 430 of a healthy individual, as recorded at a single measurement electrode referenced to an electrode that is grounded. The shape and duration of the single-ended ECAP 430 is predictable because it is a result of the ion currents produced by the ensemble of fibres depolarising and generating action potentials (APs) in response to stimulation. The evoked action potentials (EAPs) generated synchronously among a large number of fibres sum to form the ECAP 430. The ECAP 430 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The measurement circuitry 128 components may be configured to perform differential measurement of the ECAP values. Differential ECAP measurements are less subject to common-mode noise on the surrounding tissue than single-ended ECAP measurements. The measured ECAP may be parametrised by any suitable parameter(s), including, for example, an amplitude of first and second positive peaks P1 and P2, an amplitude of a negative peak N1, or a peak-to-peak amplitude (as described in International Patent Publication No. WO2015/074121, the contents of which are incorporated herein by reference). Although the embodiments described herein relate to the measurement of an ECAP, the skilled addressee will appreciate that measurement of any other type of electrical field parameter indicating a neural response may be performed alternatively, or in addition.

The relationship between the stimulus intensity (e.g. an amplitude of an applied current pulse signal) and the intensity of the neural response evoked by the stimulus (e.g. an ECAP amplitude) is represented by an activation plot, or “growth curve”. FIG. 4c illustrates an exemplary activation plot 450 for one posture of the patient 108. The activation plot 450 shows a linearly increasing ECAP amplitude for stimulus intensity values above a threshold 454 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 454 therefore reflects the field strength at which significant numbers of fibres begin to be recruited, and the increase in response intensity with stimulus intensity above the ECAP threshold reflects increasing numbers of fibres being recruited. Below the ECAP threshold 454, the ECAP amplitude may be taken to be zero. Above the ECAP threshold 354, the activation plot 450 has a positive, approximately constant slope 452 indicating a linear relationship between stimulus intensity and the ECAP amplitude.

To perform restoration of movement to spastic muscle groups, such as group 420 described herein, it is desired to achieve and maintain the neural response intensity at a target response intensity, where the target response intensity results in a desired degree of relative excitability of the stretch reflex of the muscle group (i.e., due to the recruitment of corresponding fibres). In the closed-loop mode of operation for performing SCS therapy, the stimulator 100 adjusts the intensity of an applied stimulus based on an extracted response parameter (i.e., a measured ECAP amplitude) during the therapy. The extracted neural response values are collectively referred to as a neural response signal. The target response intensity is the “recruitment target level” (RTL) mentioned above.

For example, the processor 117 may be configured to calculate an error between a target ECAP amplitude and a measured ECAP amplitude, and adjust the applied stimulus intensity to reduce the error as much as possible, such as by adding the scaled error to the current stimulus intensity. The measured neural response amplitude, and its deviation from the target response intensity, is used by the feedback loop to determine possible adjustments to the stimulus intensity parameter to maintain the neural response at the target response intensity.

The stimulator 100 is configured to apply a stimulus to the nerve fibres (e.g., the Aβ fibres or the Ia fibres) associated with the spastic muscle group as a sequence of electrical pulses according to a predefined stimulation pattern. The stimulation pattern is characterised by multiple stimulus parameters including for example, an intensity value (i.e., pulse amplitude), pulse width, number of phases, order of phases, number of stimulus electrode poles (two for bipolar, three for tripolar etc.), and stimulus rate or frequency. At least one of the stimulus parameters, for example the stimulus intensity, is controlled by the feedback loop.

In the configuration stage of step 302, a user may program the control programs 122 of stimulator 100, for example via a data exchange with user device 192. Stimulator 100 is configured to receive a set of program parameters enabling the stimulator 100 to execute the spinal activity measurement, decoding, and modulated closed-loop SCS programs (e.g., in the therapy mode of operation). The program parameters may be loaded into the memory 118 of the stimulator 100 as the clinical settings 121 by a data exchange with the user device 192, as operated by the user (e.g., a clinician).

For example, the program parameters may include a set of values defining one or more movement-specific models (e.g. expected motor pattern feature values, excitation response values, and movement phase interval values) stored in the memory 118 of module 110. The program parameters, in some examples, may also include values, such as linear regression model parameters, enabling the processor 117 to generate the time-varying RTL for CL-SCS from the excitation response values (i.e., of the neural activity patterns associated with the movement phases) in real time following or during spinal activity decoding.

As part of configuration step 302, a user may also configure the application programs of user device 192 with parameters and/or settings to enable the functionality of the neuromodulation system. For example, a user may configure the programs executed by the user device 192 to receive training spinal activity data, and/or to process the training data to train one or more spinal activity models. The process of training a movement model, as performed by a processor 194 of the device 192, may include determining expected ER values during a movement phase. Alternatively, the expected ER values may be pre-computed for one or more movement phases, and stored in the memory 196 of the device 192.

For example, to determine the ER values based on the H-reflex (as described below), and/or corresponding RTLs, a user may configure the user device 192 with parameters for generating response growth curves and/or for determining a response model to translate the ER values into corresponding RTLs for the muscle group. In some embodiments, neural response measurements obtained from prior therapy performed on the patient 108, and/or the spastic muscle(s), may be used to directly set, or inform a selection of, the stimulus parameters.

### Measuring and Decoding Spinal Activity

Referring to FIG. 3, steps 304 to 308 are performed by the stimulator 100 when operating in a therapy mode such as to provide restoration of movement to the spastic muscle group 420 of the patient 108. At step 304, the stimulator 100 is configured to measure one or more spinal activity values, also referred to as spinal activity signal (SAS) data herein, in association with an intended, or an actual, movement of the patient 108.

In some embodiments, the SAS data is generated as a series of amplitudes a₁, . . . , aₙ of n non-evoked potentials (CAPs) measured by the spinal activity electrodes over an activity detection interval Tₐ in which the patient 108 has an intention to carry out, or is actually carrying out, a movement L in association with a spastic muscle group. For example, movements such as walking and jumping may be associated with the lower limb (leg) muscle group 420 depicted in FIG. 4a. In some embodiments, controller 116 is configured to store the SAS data A={a₁, . . . , aₙ} in a data structure, such as an array, list, or table, in the memory 118 such as to enable retrieval by the processor 117.

At step 306, processor 117 of the controller 116 receives the SAS data A and processes the received values to decode the SAS. Decoding of the SAS enables the controller 116 to determine a time-varying neural activity pattern representing a degree of relative excitability of the stretch reflex of the muscle group for carrying out the movement L on muscle group 420. The neural activity pattern thereby represents a time-varying excitability of the stretch reflex associated with the muscle group in order to achieve normal (i.e., non-spastic) movement control.

### The Spinal Reflex Arc

The ability to characterize the movement L intended or performed by the patient 108 in terms of a neural activity pattern is based on the spinal reflex arc, and particularly the responses of (Ia) afferent and (a-MN) efferent fibres of the patient 108. The spinal stretch reflex arc (or monosynaptic stretch reflex) is a closed neural loop that directly connects the muscle to and from the spinal cord via the afferent and efferent pathways. The modulation of the corresponding α-MNs controls the function of the muscle.

For example, during walking, soleus muscle stretch reflexes are downregulated at heel contact to ensure force absorption from the impact while eccentric contraction of the tibialis anterior muscle and concentric contraction of the triceps surae occur, and the quadriceps α-MN excitation increases in this phase while it remains deeply depressed throughout the remaining step cycle (see Côté [2]). That is, in a normal walking movement the excitability of the stretch reflex of the associated muscle group (e.g., the flexors and extensors of the lower limb) is modulated over time (i.e., during corresponding stance and swing phases of the movement).

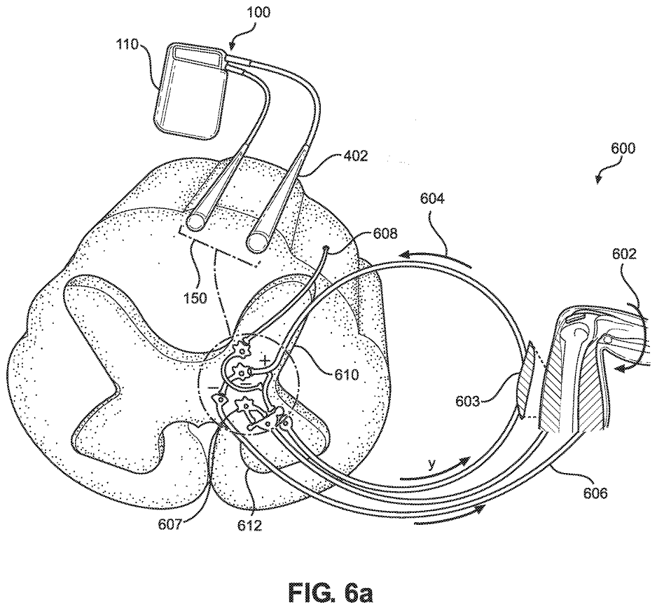

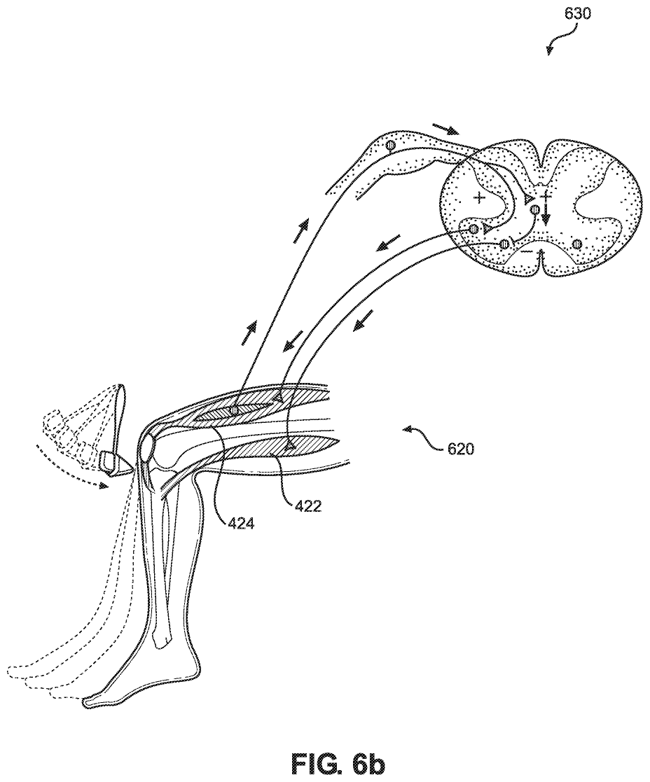

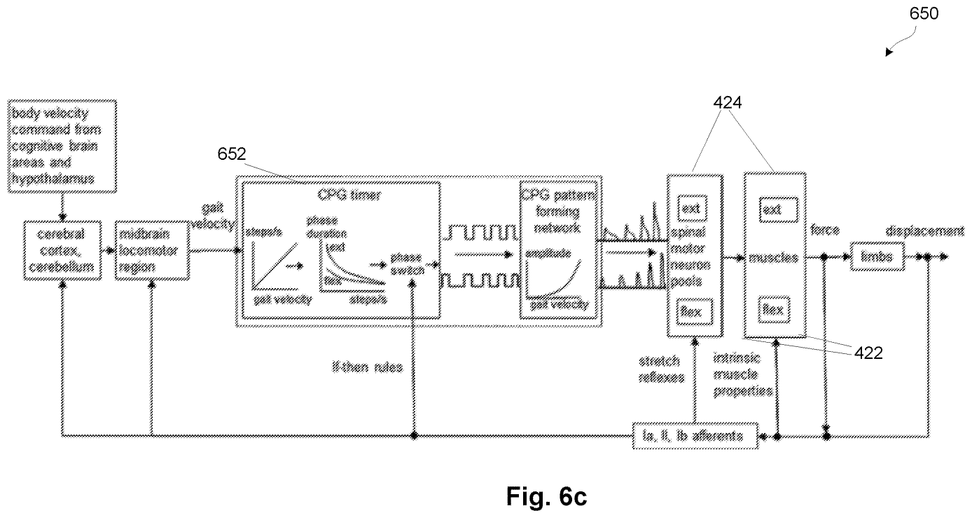

FIG. 6a illustrates the spinal reflex arc 600 in a healthy individual. FIG. 6b illustrates an example of the spinal reflex arc causing a rapid stretch 630 of the lower limb muscle group 420. The α-MNs associated with the sensory Ia afferents of the leg muscle are activated in the ventral horns of the spinal cord resulting in a delayed contraction of the muscle that was stretched. The reflex begins when the muscle spindle 603 detects a change in muscle length corresponding to a stretching of the muscle 620. In response the Ia afferent fibres 604 are activated. The Ia afferent fibres 604 transmit these sensory impulses to the dorsal horn of the spinal cord and excite the motor (muscle) efferents (α-MNs) 606 of the same muscle, thus causing the muscle to contract. At the same time, these afferents also inhibit α-MNs 606 of the antagonist or opposing muscle through inhibitory interneurons 607, causing it to relax. Cutaneous afferents from skin mechanoreceptors 608 also enter the spinal cord at the dorsal root entry zone 610 and are known to also connect with the inhibitory interneurons 607.

The spinal reflex arc may become hyper-excitable as a result of insufficient control from the brain, i.e. a lack of descending inhibition to the α-MNs. SCS of the dorsal column to stimulate the cutaneous afferent fibres such as Aβ fibres has been shown to be effective at restoring a level of inhibition to the α-MNs. On this basis, a stimulus may be applied to recruit the Aβ fibres and thereby control the degree of excitability of the stretch reflex. If the correct amount of relative excitability is provided to the stretch reflex then the muscles will experience extensions and contractions over time that are in accordance with normal behaviour of the muscle during movement (i.e., resulting in muscular activity that assists the movement intended or performed by the patient 108).

FIG. 6a shows the positioning of the electrodes of array 150 to stimulate the cutaneous receptors 608 of the Aβ fibre(s) in the dorsal column. In the described embodiments, stimulation of the Aβ fibres is achieved by a dorsal configuration of the first lead 402 of array 150, as also depicted in FIG. 4a.

The level of stimulation provided to the Aβ fibre(s) is proportional to the degree of descending inhibition provided to the stretch reflex of the muscle group. A significant consequence is the ability to relieve spasticity in a muscle, or muscle group, by supplementing a lack of descending inhibition of the stretch reflex in the muscle group. This may be achieved by stimulating the Aβ fibres on the dorsal column to a predetermined recruitment target level, based on the ability to quantify the expected degree of excitability of the stretch reflex of a muscle group without the spasticity (e.g., using a measurement of the expected H-reflex response—see International Patent Application No. PCT/AU2023/051112 as mentioned above).

The restoration of movement to a spastic muscle group is therefore hypothesized to be realizable as an extension of relieving spasticity in the muscle group, which may occur by supplementing the descending inhibition. Modulation of the amount of supplementary descending inhibition, and/or excitation, provided to the stretch reflex effectively controls a degree of relative excitability of the stretch reflex of the muscle group. That is, the stretch reflex may be modulated to a lesser or greater degree of excitability by stimulating the Aβ afferents (via the first lead 402) or the Ia afferents (via the second lead 404) associated with the muscle group with a varying level of intensity.

Quantifying the expected degree of relative excitability of the stretch reflex of a muscle group during a movement (e.g., as excitation response (ER) values) enables the activation of the Aβ or the Ia afferent fibres to be controlled by modulating a corresponding feedback target value of the CL-SCS according to an expected neural activity pattern (e.g., a series of ER values). That is, by synchronizing the amount of inhibition or excitation provided to the stretch reflex with the stages (or “phases”) of the movement, the relative excitability of the stretch reflex of the muscle group may be controlled to restore the movement.

A practical utilization of the aforementioned mechanisms is based on the use of the received spinal activity values (i.e., SAS data) to determine the intended or actual movement of an individual, and to relate the movement to a time-varying neural activity pattern describing the expected relative degree of excitability. The Central Pattern Generator (CPG) of the midbrain controls the responses of the spinal cord fibres to enable a particular form of movement (e.g., to allow co-ordination). Examples of a spinal cord-based approach to mimicking the CPG functionality are presented below, generally referred to as the process of “decoding” the received spinal activity values (SAS data).

### Representing CPG Activity Via Spinal Activity Patterns

CPGs located in the spinal cord and brainstem generate the timing and patterns needed for complete complex, rhythmic, coordinated muscle activities such as mastication, respiration, defecation, micturition and locomotion (Steuer and Guertin [4]). FIG. 6c illustrates an example of a locomotor control system 650 in mammals (from Prochazka et al. [3]). The control system 650 includes a CPG 652 that controls motor functions, such as locomotion, and which is located in the lumbar and sacral segments of the spinal cord (Steuer and Guertin [4]). The locomotor CPG 652, inactive at rest, generates a rhythmic control output by combining autonomously generated signals and control from supraspinal areas along with afferent input from body sensors activated by the movements. Supraspinal areas initiate locomotion and send a velocity command to the spinal CPG.

For example, in relation to a walking movement, the CPG output may consist of a burst of neural activity associated with flexors 422 (supporting the swing phase) and a burst of activity in extensors 424 (supporting the stance phase) (Duysens and Forner-Cordero [5]). The intrinsic stiffness of the active muscles provides immediate negative displacement feedback. Sensory input mediates negative displacement feedback and positive force feedback via spinal reflex pathways.

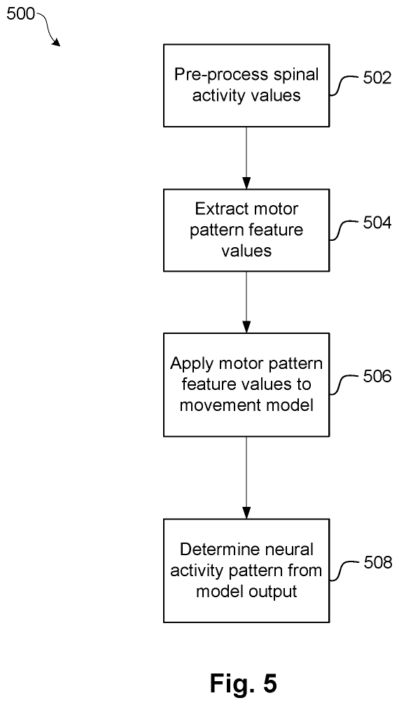

FIG. 5 illustrates a method 500 for decoding the received SAS data to generate the time-varying neural activity pattern. At step 502, the processor 117 is configured to optionally pre-process the spinal activity values a₁, . . . , aₙ by the application of one or more scaling, windowing, shifting or other pre-processing functions. In some embodiments, the pre-processing functions are applied selectively, for example as corresponding to the features that are to be subsequently extracted from the SAS data (i.e., at step 504) in order to increase the accuracy of the feature values.

At step 504, the processor 117 is configured to extract, from the SAS data a₁, . . . , aₙ, values of one or more motor pattern features (also referred to as the “motor pattern feature values”) characterizing rhythmic electrical activity associated with the intention of, or with the actual, movement L.

In the described embodiments, a sequence of motor pattern feature vectors mp is generated for corresponding time instants of spinal activity measurement (denoted as MP={mp₁, . . . , mpₙ}). The motor pattern feature vector mp includes individual motor pattern feature values which may be values of, or values derived from, the non-evoked potentials represented by the spinal activity values a₁, . . . , aₙ. For example, a subset of the spinal activity values a₁, . . . , aₙ may be processed over a particular time interval (e.g., as defined by a sliding window) to produce a feature representative of spinal activity in the interval (e.g., by averaging the activity values within the window, by taking the minimum or maximum of these values, or by comparing one or more of these values to one or more pre-determined thresholds).

In these embodiments, the representative spinal activity values may be matched to the corresponding non-evoked potentials determined for movements made by healthy individuals, the patient, or one or more individuals with the same or similar movement disorder as the patient, via analysis of corresponding SAS data determined during model training (as described below). In some embodiments, the motor pattern feature vectors mp may include only the non-evoked potentials of spinal activity measurements (or representative values of such) to indirectly characterize the rhythmic electrical activity associated with the intended or actual movement.

In other embodiments, the motor pattern features include one or more of a cycle period, a burst duration, a duty cycle, and a phase of an excitation of an individual muscle of the muscle group. These features have been shown to quantify rhythmic motor function according to the CPG model described above (Marder and Bucher [6]).

At steps 506 and 508, the controller 116 applies the extracted motor pattern feature values to one or more spinal activity models, and determines the neural activity pattern from the model output. In the described embodiments, the spinal activity models include at least one movement-specific model λ_{L} of the intended or actual movement L.

In the described embodiments, processor 194 is configured to generate the one or more spinal activity models during a training process that is conducted by user device 192 at a time prior to the application of CL-SCS therapy to patient 108. In other embodiments, the training process to generate models may be performed by an external device and the relevant parameters of the one or more models may be subsequently uploaded to the stimulator 100 (e.g., via data transfers between the external device and the user device 192, and the user device 192 and stimulator 100).

### Movement Model Training

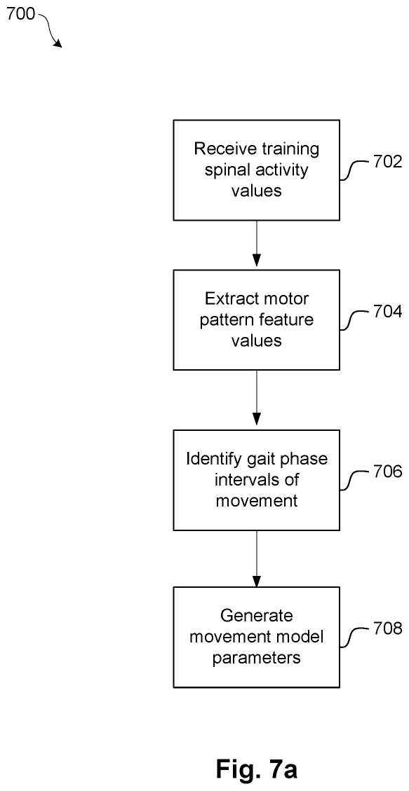

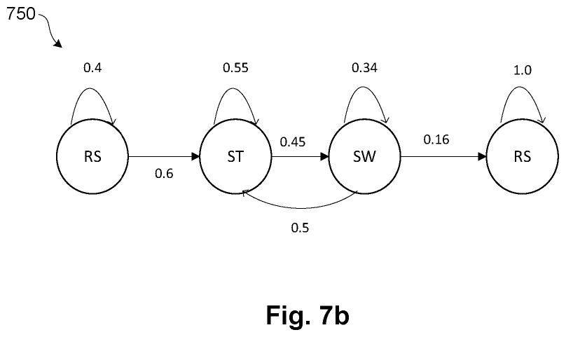

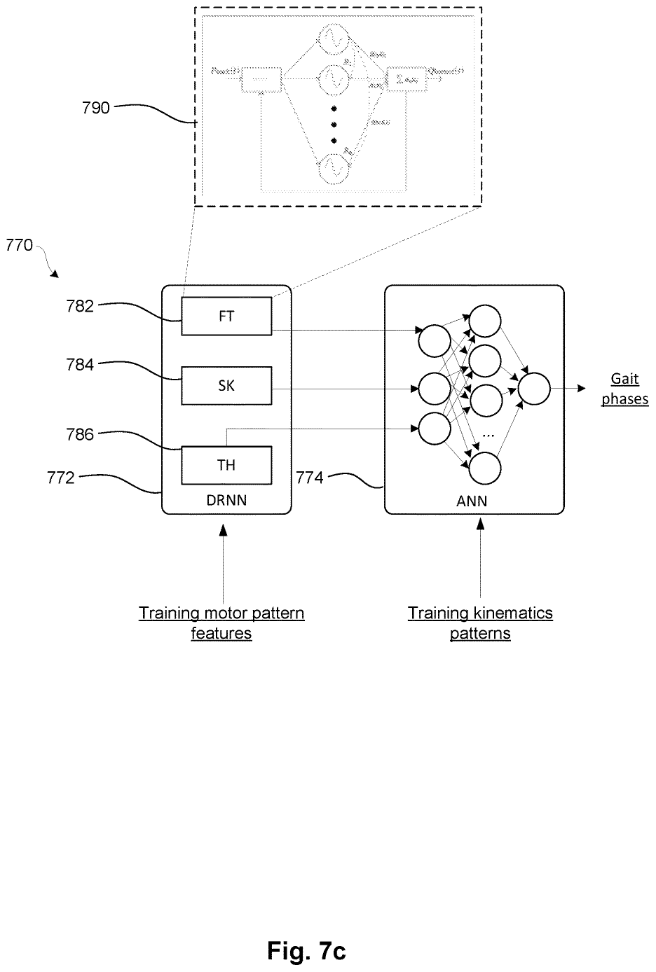

FIG. 7a illustrates an exemplary process 700 executed by user device 192 for training a spinal activity model corresponding to a given movement, for example the movement Z intended or performed by the patient 108. At step 702, processor 194 receives training data including training spinal activity values obtained from healthy individuals, the patient, or one or more individuals with the same or similar movement disorder as the patient, performing the movement L. In some embodiments, the training spinal activity values are measured by a stimulator 100 or like device coupled to each individual. The stimulator 100 or like device is configured to operate in the evaluation mode to execute a measurement operation to continuously and repeatedly measure non-evoked potentials (CAPs) at the lumbar spinal region. SAS data obtained by the stimulator 100 operating in the evaluation mode are transmitted to the user device 192, such as via channel 190.

In some configurations, the transmission of the SAS data from the stimulator 100 to the user device 192 occurs in real-time with the generation of the non-evoked potential values. In other embodiments, processor 117 may be configured to store or buffer the SAS data as evaluation data, and to transmit the stored or buffered evaluation data from the stimulator 100 to the user device 192 at a time after collection.

At step 704, processor 194 is configured to extract a training motor pattern feature vector sequence MP from the training spinal activity values. In one embodiment, a separate model λ_{L} is trained for each movement L for which restoration may be performed by the neuromodulation system. The movement model λ_{L} relates a set of expected motor pattern feature values (as determined by the model parameters) to one or more gait phases (or “movement phases”). The gait phases are specific to the movement L and the muscle group of interest and map muscular activity of the muscle group, as indicated by the CPG activity, to a time sequence over the interval in which the movement is performed. For example, λ_{L} may represent a ‘walking’ movement L for the muscle group of the lower limb muscle group 420 including extensors 424 and flexors 422. The gait phases include: a stance phase, being the interval in which a foot is on the ground; and a swing phase being the interval in which the foot is in the air, during one ‘stepping cycle’ of the walking movement. The movement-specific spinal activity model λ_{L} is also referred to as a ‘movement model’ herein.

At step 706, processor 194 identifies one or more gait phases of the movement L with respect to the training data. Spinal activity values are provided as a time series of sample values in the training data, as measured across the detection interval Tₐ. Corresponding gait phases are identified as intervals of the training data in which a phase of movement is occurring in the muscle group (i.e., ‘stance’ or ‘swing’ phases for walking).

In one implementation, the processor 194 is configured to calculate gait phases using a timing routine that mimics the function of the neural CPG timer. For example, as shown in FIG. 6c, the CPG timer routine may infer a number of steps per second of the walking motion, and use this inference to determine instants at which there is a switch between the stance and the swing phases. The number of steps (i.e., the measure of walking gait velocity) may be inferred from analysis of the non-evoked potential values of the training spinal activity values.

In some configurations, the training spinal activity values are obtained from healthy individuals. The modelling and/or mapping of spinal activity values to one or more gait phases using training data from the healthy individuals may form a ‘benchmark’ spinal activity model representing an ideal model of the movement L. In some implementations, the processor 194 adapts or modifies one or more of: the benchmark model for the movement L; and the spinal activity values measured from the patient 108 when performing, or attempting to perform, movement L. This enables the process 700 to account for a difference in the spinal activity values of the healthy individuals and the disorder-afflicted patient 108 for the movement L. In other configurations, the training data is obtained from the patient 108, or from one or more individuals with the same or similar movement disorder as the patient 108.

In some configurations, the identified one or more gait phases of the movement L may vary depending on the source of the training data. For example, the processor 194 may identify fewer gait phases, for a given movement L, in response to processing training data obtained from movement disorder afflicted individuals (e.g., the patient 108), in comparison to training data obtained from healthy individuals. The user device 192 may be configured to adjust or update one or more of the trained spinal activity models as the ability of the patient 108 to perform the movement L is restored.

In an alternative approach to determining gait phases from spinal activity (or resultant motor pattern feature) values, the processor 194 may be configured to determine the movement phases for the training data based on additional data that provides an assessment of the kinematics of the individual during training spinal activity data collection. In an example, the additional data may be provided as input received from a user of the user device 192. The user may observe the measurement of the spinal activity values from the individual during training and record indications of the position of the muscles of the muscle group as the individual is performing the movement (e.g., such that the position of the flexors and extensors can be determined). In another example, the additional data may be obtained from one or more accelerometers or other motion-tracking sensors or devices being worn by the individual. The processor 194 processes the indications of the muscle position supplied by the additional data to generate the time intervals in which gait phases of the movement occur in the time samples of the training spinal activity data.

At step 708, the processor 194 generates, by processing the training motor pattern feature values, parameters of the movement model λ_{L}. Each movement L is characterized by a time-dependent sequence of the gait phases (e.g., the repetition of stance and swing phases for walking), and model λ_{L} represents a relationship between (input) motor pattern values and the (output) gait phases, as associated with an individual performing the movement (or intending to do so).

In one example, the processor 194 executes a model training program to generate each movement model λ_{L} as a probabilistic classification model, such as a Hidden Markov Model (HMM). Each HMM λ_{L} represents the spinal activity of the movement L as a Markov process over the sequence of the motor pattern feature values (i.e., the spinal activity information) as mapped to the time-varying gait phases. For example, separate HMMs may be trained for a variety of movements including walking (λ_{L}=λ_{walk}), climbing (λ_{climb}), and jumping (λⱼᵤₘₚ). The processor 194 is configured to perform model training via executing an expectation-maximization training algorithm on the training motor pattern feature values to calculate the parameters by maximizing the log-likelihood (or posterior) for the model.

FIG. 7b illustrates an exemplary implementation of a spinal activity model λ_{L} in the form of a HMM 750 including states ST and SW corresponding to gait phases φᵢ (ST=stance φ₁, and SW=swing φ₂), where RS (φ₀) is a rest state. The observations applied to the model are motor pattern feature vectors {mp₁, . . . , mp_{Ta}} extracted from the spinal activity values {a₁, . . . , aₙ}. The walking movement is characterized by only allowing certain sequences of gait phases with greater than zero probability (as specified by a probability transition matrix). The output of HMM λ_{L}, as obtained at step 506 of FIG. 5 by applying the motor pattern feature values to λ_{L}, is an indication of a state sequence that occurs with a maximal-likelihood given the motor pattern feature observations. The maximal likelihood state sequence is translated to a sequence of the gait phases (e.g., ‘rest’, ‘swing’, ‘stance’, ‘swing’, etc.) representing the walking movement. In other embodiments, the movement-specific model may be trained with a different number of states, such as to model additional gait phases of human movement (e.g., sub-phases of ‘initial contact’, ‘loading response’, ‘mid stance’, terminal stance’, ‘pre-swing’, ‘mid swing’, and ‘terminal swing’ for walking).

Training a set of finite state machine-based models, such as HMMs, for each movement for which restoration is to be provided may enable the neuromodulation system to deliver therapy dynamically. Specifically, the controller 116, when operating in therapy mode, may selectively and autonomously determine the best movement model corresponding to the intended or actual movement by applying the motor pattern feature values to a plurality of candidate movement models (λ_{walk}, λ_{climb} and λⱼᵤₘₚ) without any other knowledge of the intended or actual movement. For example, given a set of HMM movement models {λᵢ}, the processor 194 may be configured to determine the model λ_{L} corresponding to intended or actual movement L of patient 108 by calculating the maximal observation likelihood of the sequence MP of measured motor pattern feature vectors (i.e., as extracted from the SAS data at step 504) across all models (i.e., as argmax_i p(mp|λ_i)).

Previous approaches to walking rehabilitation have modelled a brain-computer interface (BCI) as a programmable central pattern generator (PCPG) using a dynamic recurrent neural network (DRNN). The DRNN was applied to translate a brain-based activity signal generated during locomotion to control an assistive exoskeleton configured to assist the movement (Cheron et al. [7]).

In some embodiments, the processor 194 is configured to execute a model training program to generate movement-specific spinal activity model λ_{L} via a two-tier neural network classification system. FIG. 7c illustrates the exemplary implementation of the two-tier neural network classification system 770. In the first tier, a DRNN 772 is constructed to model a relationship between the time varying input of the motor pattern feature values and the kinematics of the individual's lower limb based on a set of nodes that are cyclically connected via a looping mechanism. The nodes define several individual based PCPGs for the foot (782), shank (784), and thigh (786) kinematics, as measured by an angle of elevation during movement. Each PCPG 790 is represented by a set of adaptive oscillators mutually coupled as described by Cheron [7]. In the second tier, an artificial neural network (ANN) 774 is constructed to map the kinematic outputs of the DRNN 772 to a gait phase sequence describing the movement L.

In an exemplary implementation, the DRNN 772 is constructed with 20 fully connected hidden units. The training is supervised with the training spinal activity values, involving the backpropagation through time algorithm. Training data may be obtained from healthy individuals or the patient during walking movement performed on a treadmill at varying speeds. The training data comprises the spinal activity values and output data indicating elevation angles of the lower limb body parts including the feet (FT), the shank (SK), and the thigh (TH). The ANN 774 is constructed with 10 nodes in the hidden layer, and a single output node. The number of input nodes is set to correspond to the kinematic features of the DRNN output (i.e., an input for each of the FT, SH and TH values). The weights of each ANN neuron are trained by backpropagation using a set of kinematics training data including exemplary mappings of an input vector (FT, SH, TH) to a real number j representing a gait phase of the movement L.

To perform decoding with system 770 (i.e., at step 506), the processor 117 applies motor pattern feature values to the DRNN tier 772 generating the vector (FT, SH, TH), which is input to the ANN tier 774 to generate the real valued scalar output J. Thresholding the output value J enables a binary classification of the gait phase as ‘swing’ or ‘stance’. For example, assigning training output labels of −100 and 100 to swing and stance phases for input training motor pattern feature value sequences enables a threshold of Jₜ=0 to be used to produce a binary classification of corresponding measured motor pattern feature value sequences as swing (J_t≤0) or stance (Jₜ>0).

Referring to FIG. 5, at step 508 the processor 117 is configured to determine an expected neural activity pattern based on the movement phase sequence output by the spinal activity model at step 506. In some embodiments, the controller 116 retrieves neural activity pattern data stored in the clinical data 120, clinical settings 121, or control programs 122 and processes the neural activity pattern data to map the phase sequence output to a corresponding time-varying sequence of ER values. The ER values are calculated for each gait phase present in the output sequence from a set of values determined during, or shortly following, movement model training.

For example, neural activity (excitation) patterns may be obtained from one or more of the healthy individuals or the patient from which training spinal activity values are collected. The user device 192 may be configured to generate, based on neural response intensities received in association with applied probe stimuli of varying intensity, an ER value while the individual maintains a particular gait phase (e.g., holding the lower limb muscles 422, 424 in a ‘swing’ or ‘stance’ state).

Determining a corresponding ER value for each gait phase enables the processor 194 to generate a neural activity map that is stored as data within the memory 196 (e.g., as a table or list) and subsequently transmitted to the stimulator 100. During a therapy operation, the stimulator 100 is configured to process the determined (output) phase sequences and obtain the sequence of ER values from the neural activity map, stored as a table or list structure within the memory 118.

### Graph-Based Spinal Activity Analysis

In an alternative to approaches that use pattern classification and/or machine learning (such as those described above) to train the spinal activity model, in some embodiments the processor 194 is configured to generate spinal activity graphs to capture the time-varying characteristics of the spinal activity for the movement L.

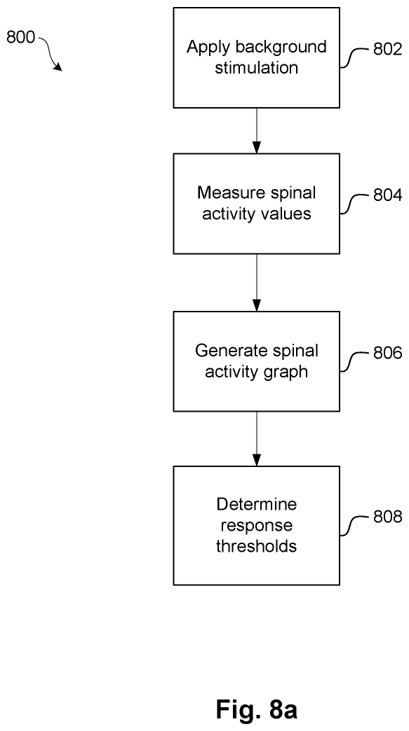

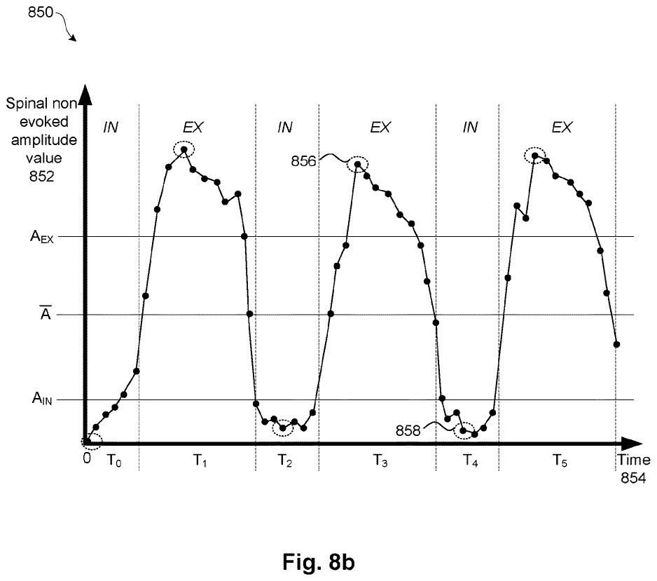

FIG. 8a illustrates an exemplary process 800 executed by user device 192 for training a spinal activity model corresponding to the movement L intended or performed by the patient 108, where the model is derived from a spinal activity graph.

At step 802, the processor 194 instructs a stimulation device, such as stimulator 100 or a similar device, to apply a constant stimulus (the “background stimulus”). The stimulation device is programmed to apply the background stimulus via the execution of a CL-SCS routine with a fixed target response intensity. In some implementations, step 802 is omitted, making the background stimulus effectively zero. At step 804, training spinal activity values are measured in accordance with the techniques described above while the patient performs or attempts to perform the movement.

At step 806, the processor 194 processes the training spinal activity values to generate a spinal activity graph. The spinal activity graph represents the variation in the amplitudes of the non-evoked potentials of the SAS data over time (i.e., as the individual progresses through the phases of the movement). FIG. 8b illustrates an exemplary spinal activity graph G (850) comprising spinal activity values a₁, . . . , aₙ (852) measured at corresponding time instants t₁, . . . , tₙ (854). The activity graph G is specific to a particular movement (e.g., walking). Processor 194 is configured to generate a spinal activity model from the activity graph G.

In the described embodiments, at step 808 the processor 194 is configured to determine representative inhibitory and excitatory response levels (thresholds) of the muscular activity by performing a thresholding function on the graph G. One or more response thresholds are calculated from the set of spinal activity values a₁, . . . , aₙ (852). In one implementation, the one or more response thresholds include: an excitation threshold above which the muscular activity represented by G is considered excitatory; and an inhibition threshold below which the muscular activity represented by G is considered inhibitory. In an alternative implementation, a single response threshold is used to determine each spinal activity value in G as excitatory (above threshold) or inhibitory (below threshold), where, for example, the single response threshold value may be derived from the excitation and/or inhibition threshold(s).

Processor 194 is configured to process the spinal activity values a₁, . . . , aₙ (852) to calculate the excitation threshold and the inhibition threshold by detecting non-evoked potential amplitudes corresponding to local maxima (856) and local minima (858) respectively in G. For example, processor 194 may determine that one or more of spinal activity values a₁, . . . , aₙ correspond to local maxima (minima) by comparing samples within a time window of a pre-determined size, where the maxima (minima) values are larger (smaller) than all adjacent values within the window.

The processor 194 determines the excitation and inhibition thresholds by processing the set of local maxima {a}ₘₐₓ and minima {a}ₘᵢₙ values. In one example, the excitation A_{EX} and inhibition A_{IN} thresholds are calculated as the average of the local maxima and minima sets, as: A_{EX}=\overline{{a}ₘₐₓ}−B and A_{IN}=\overline{{a}ₘᵢₙ}+β, where β≥0 is an offset value. The offset value provides a tolerance enabling the consideration of additional spinal activity values as representing excitation or inhibition. The excitation A_{EX} and inhibition A_{IN} thresholds are the parameters of the graph-based spinal activity model λ_{L}.

The spinal activity model λ_{L} is therefore generated by combining the spinal activity graph G with the determined gait phase sequence (i.e., the indications of the phase {φ₁, φ₂, φ₁, . . . } and the corresponding time intervals {[t₀, t_{z1}], [t_{z2}, t_{z3}], . . . }).

In another implementation, spinal activity model λ_{L} is generated by combining the spinal activity graph G with the indications of the excitation and inhibition periods {IN, EX, IN, . . . } and the corresponding time intervals {T₀, T₂, . . . } and {T₁, T₃, . . . }, and the threshold values A_{EX} and A_{IN}.

The spinal activity model λ_{L} is utilized by the processor 117 (i.e., at steps 506 and 508 of decoding spinal activity measured from patient 108) to generate neural activity pattern values based on the movement phase sequence. As described above, the neural activity pattern values (e.g. ER values) may be obtained from the gait phase sequence by a neural activity map (as programmed into the stimulator 100).

In one implementation of step 506 of the method 500, the processor 117 forms the spinal activity graph G from the spinal activity values a₁, . . . , aₙ of the spinal activity signal. The processor 117 then, based on the representative inhibitory and excitatory response levels (i.e., excitation and inhibition thresholds A_{EX} and A_{IN}), determines one or more time periods of relative excitation or inhibition associated with performing the movement. An exemplary process for deriving the time periods of relative excitation and inhibition is as follows: (i) representative inhibitory and excitatory response levels A_{EX} and A_{IN} are averaged to generate a threshold Ā; (i) starting at i=1 determine a new excitation (inhibition) time period if aᵢ>Ā (aᵢ≤Ā); (ii) if the current time period is excitation (inhibition), then increment i until aᵢ≤Ā (aᵢ>Ā) where a zero-crossing of the spinal activity value is achieved relative to Ā; (iii) commence a new inhibition (excitation) time period at a time t∈[tᵢ₋₁, tᵢ] where the zero-crossing occurred at tᵢ; (iv) repeat steps (ii) to (iv) for all spinal activity values a₁, . . . , aₙ. By executing the above steps, the processor 117 is configured to generate time intervals corresponding to the excitation and inhibition periods of movement.

FIG. 8b illustrates excitation (EX) periods T₁, T₃, and T₅ and inhibition (IN) periods T₀, T₂, and T₄ determined in accordance with the above process and based on the excitation and inhibition thresholds A_{EX} and A_{IN}. At step 508, the processor 194 determines the expected neural activity pattern for the movement by processing the one or more periods of relative excitation or inhibition.

In one implementation of step 506, the processor 194 maps the determined periods of relative excitation and inhibition to one or more gait phases of the movement. For example, in the case of a walking movement, the interval defined by a pair of consecutive excitation and inhibition periods may be mapped to swing and stance phase intervals. As illustrated in FIG. 8b, the phase intervals may not necessarily coincide with the excitation and inhibition intervals.

In one implementation of step 508, the processor 117 determines the time-varying neural activity pattern values based on a selection of either the excitation or the inhibition threshold value defined by the spinal activity model λ_{L}. For example, for times within a period of excitation identified in the graph, the processor 117 selects threshold A_{EX} as the neural activity pattern value. For times within a period of inhibition identified in the graph, the processor 117 selects threshold A_{IN} as the neural activity pattern value.

This approach is based on an inferred relationship between the amplitude of the cutaneous afferent response (i.e., the neural response intensity of the CL-SCS, as described below) and the amplitude of the non-evoked potentials (i.e., the spinal activity values a₁, . . . , aₙ) of graph G. In some embodiments, the processor 117 applies scaling, transformation or other processing operations to the threshold values A_{EX} and A_{IN} to generate the neural activity pattern sequence (e.g. the ER values).

The embodiments described above determine spinal activity models based on training data obtained from healthy individuals, the patient, or other individuals afflicted with the movement disorder, performing the movement. In some other embodiments, training spinal activity data is obtained from the individuals performing a plurality of movements involving the muscle group. For example, movements involving muscle group 420 may include walking, jumping, climbing and other related movements involving activity of the flexor 422 and extensor 424 muscles.

In some configurations, the processor 194 of device 192 is configured to process the training data including spinal activity values measured during the plurality of movements to generate a single generalized (i.e., non-movement-specific) spinal activity model λ. By applying a series of motor pattern feature vectors MP, as extracted from the measured SAS data, to model λ the processor 117 generates an arbitrary sequence of gait phase outputs that may correspond to any movement associated with the muscle group. This is advantageous in enabling a generalized means of restoring movement to a spastic muscle group of patient 108.

In accordance with the above described approaches, decoding a spinal activity signal representing an intention to perform, or the actual performance of, a rhythmic motor pattern (i.e., a walking movement) thereby enables the determination of the corresponding expected time-varying neural activity pattern for the muscle group. This approach thereby mimics the operation of the CPG and restores motor control to spastic and movement-disorder-afflicted muscles without requiring the capture or analysis of brain signals (i.e., as a purely spinal cord based approach).

### Performing Modulated Closed-Loop SCS

Referring to FIG. 3, at step 308 the stimulator 100 is configured to perform SCS therapy to restore movement to the spastic muscle group of the patient 108. This therapeutic process is referred to as “restoration of movement” below for simplicity. The applied SCS is modulated in a closed loop according to the expected time-varying neural activity pattern for the muscle group, as determined by decoding the measured spinal activity values (i.e., at step 306).

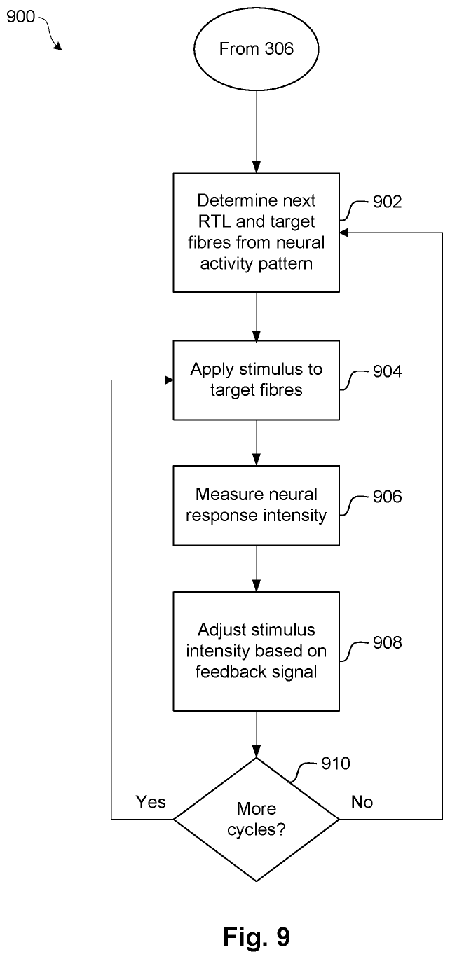

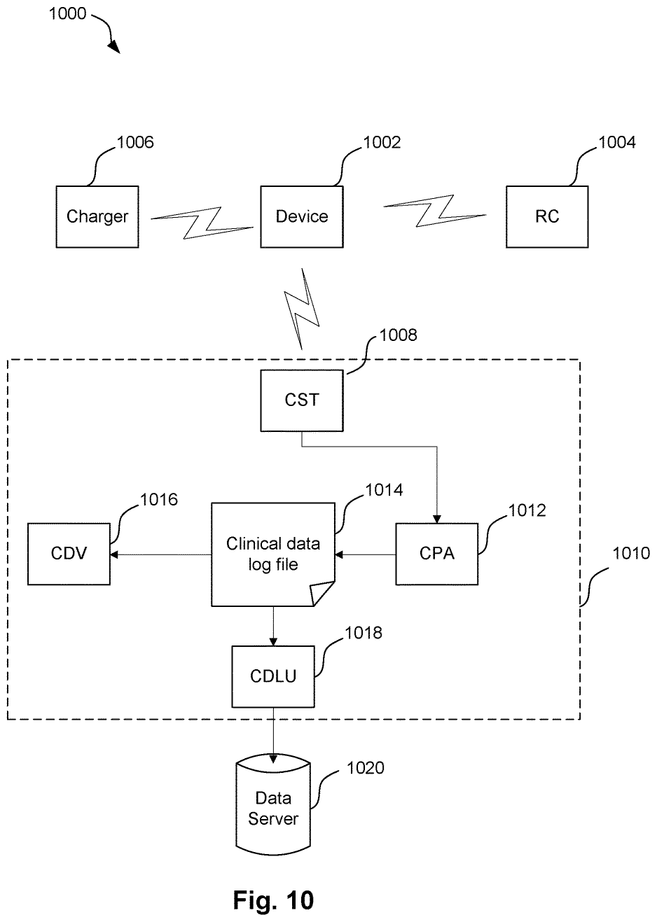

FIG. 9 illustrates a method 900 of performing modulated SCS via a neuromodulation device. FIG. 10 is a block diagram of a neuromodulation system 1000 configured to perform the method 900. In one example, the neuromodulation device 1002 of FIG. 10 is implemented as the stimulator 100 of FIG. 1, implanted within the patient 108 (not shown). In this mode of operation, stimulator 100 is a Closed-Loop Neural Stimulation (CLNS) device.

The neuromodulation device 1002 is connected wirelessly to a remote controller (RC) 1004. The remote controller 1004 is a portable computing device that provides the patient 108 with control of the closed-loop SCS therapy by providing selective control over at least some of the functionality of the neuromodulation device 1002, including: enabling or disabling closed-loop SCS according to a movement restoration program; and selection of a movement restoration program from the control programs stored on the neuromodulation device 1002 (e.g., control programs 122 of stimulator 100).

In some embodiments, the movement restoration programs of the neuromodulation device 1002 are configured by an external computing device, such as the user device 192, and are uploaded to the neuromodulation device 1002 by a user or clinician. In some embodiments, the neuromodulation device 1002 is configured to commence, continue and/or cease therapeutic closed-loop SCS autonomously and independently of instructions or signals received from the user device 192, or any other computing device, of the therapy system 1000.

Neuromodulation system 1000 includes a charger 1006 configured to recharge a rechargeable power source of the neuromodulation device 1002. The recharging is illustrated as wireless in FIG. 10 but may be wired in alternative implementations.

The neuromodulation device 1002 is wirelessly connected to a Clinical System Transceiver (CST) 1008. The wireless connection may be implemented as the communications channel 190 of FIG. 1. The CST 1008 acts as an intermediary between the neuromodulation device 1002 and a Clinical Interface (CI) 1010, to which the CST 1008 is connected. A wired connection is shown in FIG. 10, but in other implementations, the connection between the CST 1008 and the CI 1010 is wireless.

The CI 1010 may be implemented as the user device 192 of FIG. 1. The CI 1010 is configured to program the neuromodulation device 1002 and recover data stored on the neuromodulation device 1002. This configuration is achieved by program instructions collectively referred to as the Clinical Programming Application (CPA) and stored in an instruction memory of the CI 1010. For example, the CPA may specify particular settings and/or operational modes of the neuromodulation device 1002 according to the context of the therapeutic movement restoration that is to be provided by the system 1000.

To effect suitable SCS therapy, neuromodulation device 1002 may deliver tens, hundreds or even thousands of therapeutic stimuli per second, for many hours each day. The feedback loop may operate for most or all of this time, by obtaining neural response recordings following every therapeutic stimulus, or at least obtaining such recordings regularly. Each recording generates a feedback variable such as a measure of the amplitude of the evoked neural response, which in turn results in the feedback loop changing at least one stimulus parameter for a following therapeutic stimulus. Neuromodulation device 1002 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 1002 transmits data, e.g. via telemetry module 114, to a CPA 1012 installed on the CI 1010. The data can be grouped into two main sources: (1) Data collected in real time during a programming session; (2) Data downloaded from a stimulator after a period of non-clinical use by a patient. The CPA collects and compiles the data into a clinical data log file.

All clinical data transmitted by the neuromodulation device 1002 may be compressed by use of a suitable data compression technique before transmission by telemetry module 114 and/or before storage into the memory 118 to enable storage by neuromodulation device 1002 of higher resolution data. This higher resolution allows neuromodulation device 1002 to provide more data for post-analysis and more detailed data mining for events during use. Alternatively, compression enables faster transmission of standard-resolution clinical data.

The clinical data log file 1014 is manipulated, analysed, and efficiently presented by a clinical data viewer (CDV) 1016 for field diagnosis by a clinician, field clinical engineer (FCE) or the like. CDV 1016 is a software application installed on the CI 1010. In one implementation, CDV 1016 opens one Clinical Data Log file 1014 at a time. CDV 1016 is intended to be used in the field to diagnose patient issues and optimise therapy for the patient. CDV 1016 may be configured to provide the user or clinician with a summary of neuromodulation device usage, therapy output, and errors, in a simple single-view page immediately after log files are compiled upon device connection.

Clinical Data Uploader (CLDU) 1018 is an application that runs in the background on the CI 1010, that uploads files generated by the CPA 1012, such as the clinical data log file 1014, to a data server 1020. Database Loader (not shown) is a service which runs on the data server and monitors the patient data folder for new files. In response to Clinical Data Log file 1014 being uploaded by Clinical Data Uploader 1018, a database loader extracts the data from the file and loads the extracted data to a database of the data server 1020 (not shown).

The data server 1020 further contains one or more APIs, such as for example a data analysis web API, which provide data for third-party analysis such as by one or more computing devices located remotely from the data server 1020. The ability to obtain, store, download and analyse large amounts of neuromodulation data in accordance with the methods described herein is advantageous in: improving patient outcomes in difficult conditions; enabling faster, more cost effective and more accurate troubleshooting and patient status; and enabling the gathering of statistics across patient populations for later analysis, with a view to diagnosing aetiologies and predicting patient outcomes.

Referring to FIG. 9, in the described embodiments the method 900 for modulated closed-loop SCS restoration of movement is performed by stimulator 100 and user device 192, as depicted in FIG. 1. In other embodiments, another CLNS device may be configured to perform the method 900.

Restoration of movement is achieved by ensuring that the modulation of the stimulus intensity is synchronized with the intended or actual movement (i.e., that it is appropriate to enable muscular activity assisting the movement). The time-varying modulation of the stimulus intensity is based on a corresponding expected degree of relative excitability of the stretch reflex of the muscle group. The neural activity pattern represents the expected degree of relative excitability, for example, as a series of excitation response (ER) values ER₁, . . . , ER_{T} at corresponding time instants t₁, . . . , t_{T}.

At step 902, the controller 116 is configured to determine the target afferent fibre type to be stimulated based on the determined ER value ER_{d} extracted from the neural activity pattern. In one implementation, if ER_{d} is less than an ER threshold, the stretch reflex needs to be inhibited. The target afferent fibres are therefore the Aβ afferents, which may be stimulated via the first lead 402. If ER_{d} is greater than the ER threshold, the stretch reflex needs to be excited. The target afferent fibres are therefore the Ia afferents, which may be stimulated via the second lead 404.

At step 902, the controller 116 is configured to determine an RTL that varies in accordance with corresponding values of the neural activity pattern. The controller 116 is configured to output an RTL given an input ER value ER_{d}∈{ER₁, . . . , ER_{T}} (the “determined ER value”) extracted from the neural activity pattern. The RTL is an ECAP amplitude value or another electrical field parameter value that is measurable by the neuromodulation system to perform closed-loop SCS.

In a single execution of step 308, one or more cycles of SCS are performed for each RTL generated from the corresponding ER_{d} value (i.e., as part of the closed loop between steps 904 and 908 described below).

In some embodiments, the controller 116 determines the RTL from the corresponding ER_{d} value at step 902 by executing a mapping function. The determination of the RTL, as performed by the mapping function, may depend on the determined target fibre type. In one implementation, the controller 116 derives an ER-RTL mapping by retrieving the RTL from a response table that is specific to the target fibre type and is stored in local memory (e.g., as part of a control program 122). In some configurations, the response table is programmed into the stimulator 100 by the user device 192, which is configured to determine target ECAP values V (“validated ECAP values”) for each of a series of candidate ER values representing a degree of stretch reflex excitability in the muscle group. The user device 192 is configured to determine the validated ECAP values by conducting one or more experimental trials on an evaluation group of patients.

To determine the ER-RTL mapping from the programmed response table, the controller 116 determines the index of the recorded ER value ER₁, . . . . ERₚ (e.g., the ER values of the respective evaluation group patients) within the table that is closest to the determined ER value ER_{d} generated for patient 108.

In another implementation, the controller 116 determines the RTL from an ER value using a response model that is specific to the target fibre type, such as a linear regression function or a pattern classifier. The response model is trained to generate the RTL as an output for a given ER value input. For example, a set of ER values and corresponding ECAP values may be obtained from healthy individuals or the patient enabling the training of a response model to produce the RTL based on the ER value, or the difference between this value and an expected value.

The above approaches are suitable for embodiments in which the ER values are derived from the H-reflex of the of the muscle group, as described in the above-mentioned International Patent Application No. PCT/AU2023/051112. In other embodiments, determining the RTL involves scaling the magnitude of the non-evoked potentials. For example, a scaling factor may be determined by empirical evaluations performed to relate the magnitude of the non-evoked potentials to those of the neural response to an applied stimulus.

At step 904, stimulator 100 applies a stimulus of an initial intensity to the spinal cord to stimulate the one or more target afferent (Aβ or Ia) fibres as determined in step 902. Pulse generator 124 applies the stimulus via stimulus electrodes of the first lead 402 if the target afferents are the Aβ fibres, or the second lead 404 if the target afferents are the Ia fibres. The initial stimulus intensity value is specified by a control program 122 such as a closed-loop therapy routine with patient and or therapy specific parameters determined by the data 120 and clinical settings 121.

At step 906, the stimulator 100 measures a neural response evoked by the application of the stimulus. The intensity of the evoked response (e.g., the magnitude of the measured amplitude of the evoked response signal) provides a measure of the recruitment of the target fibres. A linear response curve between the applied therapeutic stimulus intensity and the response intensity may be assumed as shown in FIG. 4c. The response intensity values are measured by measurement circuitry 128 from an electrical signal sensed via measurement electrodes of the first lead 402 if the target afferents are the Aβ fibres, or the second lead 404 if the target afferents are the Ia fibres. In one implementation, the response intensity comprises a peak-to-peak ECAP amplitude. The measured response intensity values are processed by the controller 116 to perform closed-loop time-varying modulation of the stimulus intensity.

The controller 116 is configured to determine a feedback signal representing a difference between one or more values of the neural response intensity and the RTL. The controller 116 compares the measured response intensity to the RTL and provides an indication of the difference as an error value. The error value is input into a feedback unit of the controller 116.

At step 908, the feedback unit of controller 116 adjusts an intensity of the therapeutic stimulus in response to the feedback signal with the aim of maintaining a measured neural response intensity equal to the RTL. The feedback unit of controller 116 adjusts the therapeutic stimulus intensity parameter to minimise the error value. In one implementation, the controller 116 utilises a first order integrating function in order to provide suitable adjustment to the therapeutic stimulus intensity parameter. In some embodiments, the feedback unit of controller 116 may be configured to adjust the therapeutic stimulus intensity based on one or more other settings or parameters. For example, the feedback unit may be configured to a comprise a gain parameter by which to scale the feedback signal based on the clinical settings 121, such as to account for patient specific tolerances and/or sensitivities.

In some implementations, the re-application of the therapeutic stimulus with the adjusted stimulus intensity is controlled by a stimulus clock operating at a stimulus frequency (e.g. 60 Hz) and a sample clock for sampling the response signal (for example, operating at a sampling frequency of 10 kHz). On the next stimulus clock cycle, the stimulator 100 outputs a therapeutic stimulus in accordance with the adjusted therapeutic stimulus intensity. Accordingly, there is a delay of one stimulus clock cycle before the therapeutic stimulus intensity is updated in light of the feedback signal.

The controller 116 is configured to repeatedly apply the stimulus at the adjusted intensity values to achieve modulated closed-loop control of the therapeutic stimulation of the target afferent fibres. At step 910, the closed-loop control is achieved by determining whether to repeat steps 904 to 908. In one implementation, the controller 116 implements closed-loop control to perform a pre-determined threshold number of stimulus cycles (iterations of steps 904 to 908) for each determined RTL, where the threshold number is determined from the program data 120 or clinical settings 121. Alternatively, or in addition, the controller 116 may be configured to repeatedly iterate steps 904 to 908 until the feedback signal is within an error tolerance value ϵ>0.

Following the completion of the closed-loop processing for the last ER value ER_{T}, in some embodiments the neural activity pattern is updated by repetition of the decoding step 306 (i.e., as part of a closed loop between steps 304 and 308, as depicted in FIG. 3).

Modulation of the SCS is therefore advantageously achieved in two ways. First, the target ECAP amplitude value (i.e., the RTL) used to generate the feedback signal is time-varying in that the RTL is determined from the corresponding time-varying neural activity pattern. Second, the neural activity pattern is itself updated by the repetition of the spinal activity measurement and decoding steps (i.e., in steps 304 and 306 of the method 300, as shown in FIG. 3).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

### REFERENCES

[1] J. Parker, B Dietz. Spinal cord stimulation for the relief of spasticity from cerebral palsy. Healthcare Technology Letters, 2020—IET. 7(3) 93-97.
[2] Côté et al. Spinal Control of Locomotion: Individual Neurons, Their Circuits and Functions, 2018, Frontiers in Physiology, 9. https://doi.org/10.3389/fphys.2018.00784
[3] Prochazka, A., Gosgnach, S., Capaday, C., Geyer, H., 2017. Neuromuscular models for locomotion. Bioinspired Legged Locomotion. Elsevier, pp. 401-453.
[4] Steuer, I., Guertin, P. A., 2019. Central pattern generators in the brainstem and spinal cord: an overview of basic principles, similarities and differences. Rev. Neurosci. 30, 107-164.
[5] Duysens, J., Forner-Cordero, A., 2019. A controller perspective on biological gait control: Reflexes and central pattern generators. Annu. Rev. Control 48, 392-400.
[6] Marder, E., Bucher, D., 2001. Central pattern generators and the control of rhythmic movements. Curr. Biol. 11, R986-R996.
[7] Cheron et al. From Spinal Central Pattern Generators to Cortical Network: Integrated BCI for Walking Rehabilitation, 2011, Neural Plasticity, Volume 2012.

LABEL LIST

| | |
|---|---|
| stimulator | 100 |
| patient | 108 |
| module | 110 |
| battery | 112 |
| telemetry module | 114 |
| controller | 116 |
| processor | 117 |
| memory | 118 |
| program data | 120 |
| clinical settings | 121 |
| control programs | 122 |
| pulse generator | 124 |
| electrode selection module | 126 |
| measurement circuitry | 128 |
| electrode array | 150 |
| communications channel | 190 |
| user device | 192 |
| processor | 194 |
| memory | 196 |
| method | 300 |
| step | 302 |
| step | 304 |
| step | 306 |
| step | 308 |
| ECAP threshold | 354 |
| DC | 401 |
| first lead | 402 |
| second lead | 404 |
| Ia afferent fibres | 408 |
| efferent fibres | 410 |
| limb muscle group | 420 |
| flexors | 422 |
| extensors | 424 |
| ECAP | 430 |
| activation plot | 450 |
| constant slope | 452 |
| ECAP threshold | 454 |
| method | 500 |
| step | 502 |
| step | 504 |
| step | 506 |
| step | 508 |
| spinal reflex arc | 600 |
| muscle spindle | 603 |
| Ia afferent fibres | 604 |
| motor neurons | 606 |
| inhibitory interneurons | 607 |
| skin mechanoreceptors | 608 |
| dorsal root entry zone | 610 |
| muscle | 620 |
| rapid stretch | 630 |
| control system | 650 |
| locomotor CPG | 652 |
| exemplary process | 700 |
| step | 702 |
| step | 704 |
| step | 706 |
| step | 708 |
| HMM | 750 |
| system | 770 |
| DRNN | 772 |
| ANN | 774 |
| foot | 782 |
| shank | 784 |
| thigh | 786 |
| PCPG | 790 |
| process | 800 |
| step | 802 |
| step | 804 |
| step | 806 |
| step | 808 |
| spinal activity graph | 850 |
| local maxima | 856 |
| local minima | 858 |
| method | 900 |
| step | 902 |
| step | 904 |
| step | 906 |
| step | 908 |
| step | 910 |
| therapy system | 1000 |
| neuromodulation device | 1002 |
| remote controller RC | 1004 |
| charger | 1006 |
| CST | 1008 |
| CI | 1010 |
| CPA | 1012 |
| clinical Data Log file | 1014 |
| CDV | 1016 |
| clinical Data Uploader | 1018 |
| data server | 1020 |

1. A method for restoring movement to a spastic muscle group, the method comprising:
measuring one or more spinal activity values in association with a movement;
decoding the measured spinal activity values to determine a time-varying neural activity pattern; and
performing spinal cord stimulation (SCS) of one or more afferent fibres associated with the muscle group to enable muscular activity assisting the movement, wherein the SCS is modulated according to the neural activity pattern.

2. The method of claim 1, wherein measuring the spinal activity values comprises measuring non-evoked potentials at the spinal cord.

3. The method of claim 2, wherein the non-evoked potentials are sensory afferent responses associated with the movement as it occurs.

4. The method of claim 2, wherein the non-evoked potentials are efferent potentials associated with the movement.

5. The method of claim 1, wherein measuring the spinal activity values comprises measuring activity using an accelerometer.

6. The method of claim 1, wherein the neural activity pattern is a time series of values representing a degree of relative excitability of a stretch reflex of the muscle group at discrete sample times of the movement.

7. The method of claim 6, wherein the neural activity pattern is a sequence of excitation response (ER) values.

8. The method of claim 7, wherein the ER values are determined from an induced H-reflex response of the muscle group.

9. The method of claim 8, wherein the ER values are determined from a growth curve of the H-reflex response of the muscle group.

10. The method of claim 1, wherein decoding the measured spinal activity values comprises:
(i) extracting, from the spinal activity values, one or more motor pattern feature values associated with the movement; and
(ii) determining the neural activity pattern by applying the one or more motor pattern feature values to one or more spinal activity models.

11. The method of claim 10, wherein the one or more motor pattern feature values include corresponding spinal activity values.

12. The method of claim 10, wherein motor pattern features include one or more of: a cycle period, a burst duration, a duty cycle, and a phase of an excitation of an individual muscle of the muscle group.

13. The method of claim 10, wherein at least one of the spinal activity models is a movement-specific model trained with a set of training spinal activity values obtained from individuals performing the movement.

14. The method of claim 13, wherein training the movement-specific model comprises:
identifying one or more gait phases of the movement;
extracting training motor pattern feature values from the training spinal activity values; and
generating, from the training motor pattern feature values, model parameters representing expected motor pattern feature values for the one or more gait phases of the movement.

15. The method of claim 14, wherein the movement-specific model is a two-tier neural network classifier with parameters generated by processing an input sequence of the training motor pattern feature values.

16. The method of claim 14, wherein the movement-specific model is a hidden Markov model with parameters generated by processing an input sequence of the training motor pattern feature values.

17. The method of claim 13, wherein training the movement-specific model comprises:
processing the training spinal activity values to generate a spinal activity graph; and
determining representative inhibitory and excitatory response levels from the spinal activity graph.

18. The method of claim 17, wherein the representative inhibitory and excitatory response levels are threshold values determined by identifying local maxima and local minima in the spinal activity graph.

19. The method of claim 17, wherein determining the neural activity pattern comprises:
determining, based on the representative inhibitory and excitatory response levels, one or more periods of relative excitation or inhibition associated with performing the movement.

20. The method of claim 19, wherein determining the neural activity pattern further comprises processing the one or more periods of relative excitation or inhibition to determine one or more gait phases of the movement.

21. The method of claim 1, wherein performing the spinal cord stimulation includes:
(i) determining a recruitment target level (RTL) by processing the neural activity pattern;
(ii) applying a stimulus to the spinal cord to stimulate the one or more afferent fibres associated with the muscle group;
(iii) measuring an intensity of a neural response evoked by the stimulus; and
(iv) adjusting an intensity of a subsequent stimulus applied by step (ii) based on a feedback signal representing a difference between the measured neural response intensity and the determined RTL,
wherein steps (i) to (iv) are repeated such that the determined RTL varies over time in accordance with corresponding values of the neural activity pattern.

22. The method of claim 21, wherein the steps (ii) to (iv) are repeated over a number of cycles for each determined RTL of step (i), wherein the number of cycles is a predetermined threshold number.

23. The method of claim 21, wherein the steps (ii) to (iv) are repeated in one or more cycles until the feedback signal is within an error tolerance value for each determined RTL of step (i).

24. The method of claim 21, wherein, in response to the neural activity pattern comprising one or more excitation response values determined from an induced H-reflex response of the muscle group, determining the RTL involves using the determined excitation response values as input to a response model.

25. The method of claim 24, wherein the response model is a linear regression function that is specific to the one or more afferent fibres.

26. The method of claim 21, wherein the one or more afferent fibres are determined from the neural activity pattern.

27. A system for restoring movement to a spastic muscle group, the system comprising:
a stimulator including an electrode array and a pulse generator, the stimulator configured to:
measure one or more spinal activity values in association with a movement; and
apply, via the electrode array, stimuli to the spinal cord to stimulate one or more afferent fibres associated with the muscle group; and
a processor configured to:
decode the one or more spinal activity values to determine a time-varying neural activity pattern; and
control the stimulator to apply the stimuli to the one or more afferent fibres to enable muscular activity assisting the movement, wherein the stimuli are modulated according to the neural activity pattern.

28. The system of claim 27, wherein the electrode array comprises one or more leads implanted proximate to the afferent fibres.

29. The system of claim 28, wherein the one or more afferent fibres comprise Aβ afferent fibres.

30. The system of claim 29, wherein one of the one or more leads is implanted above the midline of the dorsal column.

31. The system of claim 28, wherein the one or more afferent fibres comprise Ia afferent fibres.

32. The system of claim 31, wherein one of the one or more leads is implanted over the dorsal roots of the dorsal column.

33. The system of claim 27, wherein the processor is part of the stimulator.
